(19) United States
(12) Patent Application Publication
(10) Pub. No.: US 2026/0112031 A1
(43) Pub. Date: Apr. 23, 2026

(54) HARMONIZATION OF DIFFUSION MAGNETIC RESONANCE IMAGING

(71) Applicant: Imeka Solutions Inc., Sherbrooke (CA)

(72) Inventors: Pierre-Marc Jodoin, Eastman (CA); Gabriel Girard, Sherbrooke (CA); Manon Edde, Sherbrooke (CA); Félix Dumais, Sherbrooke (CA); Matthieu Dumont, Sherbrooke (CA); Guillaume Theaud, Sherbrooke (CA); Yoan David, Sherbrooke (CA); Jean-Christophe Houde, Sherbrooke (CA); Maxime Descoteaux, Magog (CA)

(21) Appl. No.: 19/364,383

(22) Filed: Oct. 21, 2025

Related U.S. Application Data
(60) Provisional application No. 63/709,998, filed on Oct. 21, 2024.

(51) Int. Cl. G06T 7/00; G06T 7/174; G06T 11/00; G16H 20/10

(52) U.S. Cl. CPC G06T 7/0012; G06T 7/174; G06T 12/10; G06T 12/20; G06T 12/30; G16H 20/10; G06T 2207/10092; G06T 2207/30016; G06T 2207/30168; G06T 2210/41; G06T 2211/424

(57) ABSTRACT

A computer system that performs pairwise harmonization is described. During operation, the computer system may receive information specifying magnetic-resonance diffusion measurements, where the magnetic-resonance diffusion measurements are associated with at least an individual and a first site. Then, the computer system may perform the pairwise harmonizing of the magnetic-resonance diffusion measurements based at least in part on target magnetic-resonance diffusion measurements, where the target magnetic-resonance diffusion measurements are associated with a population and one or more target sites, and the magnetic-resonance diffusion measurements and the target magnetic-resonance diffusion measurements may be acquired using different magnetic-resonance scanners. Next, the computer system may perform quality control on the pairwise harmonized magnetic-resonance diffusion measurements, where the quality control may include comparing the pairwise harmonized magnetic-resonance diffusion measurements associated with the first site and the one or more target sites.

### CROSS REFERENCE TO RELATED APPLICATIONS

This application claims priority under 35 U.S.C. 119 (e) to: U.S. Provisional Application Ser. No. 63/709,998, “Harmonization of Diffusion Magnetic Resonance Imaging,” filed on Oct. 21, 2024, by Pierre-Marc Jodoin, et al, the contents of which are herein incorporated by reference.

### FIELD

The described embodiments relate to processing of medical images. Notably, the described embodiments relate to techniques for harmonizing diffusion magnetic resonance imaging (MRI).

### BACKGROUND

Neuroimaging studies increasingly utilize multicentric diffusion MRI (dMRI) data alongside normative modeling. Normative modeling is a framework designed to map the general trend and variability of a reference population, facilitating statistical analyses for individual participants with specific diseases. The detection of subtle brain variations necessitates large datasets to ensure robust and sufficient statistical power. However, it is well-established that dMRI data are influenced by site-specific acquisition biases, making cross-scanner and cross-protocol comparisons of dMRI data unreliable without appropriate data harmonization. Such discrepancies, which are sometimes referred to as ‘batch effects,’ typically arise from various technical variabilities, including software updates, scanner drift, MRI manufacturer differences, acquisition protocols, field strength, and/or hardware variability.

The objective of harmonization is to derive a transfer function between two or more sites to eliminate batch effects while preserving covariate expressions, such as age and sex. Although various harmonization techniques have been proposed, many are tailored to specific contexts and, therefore, may not be suitable for routine clinical practice, which comes with its own set of challenges. For example, data from day-to-day clinical practice often originate from patients with a broad spectrum of diseases and symptoms, unlike clinical studies and academic research that typically involve carefully selected participants with consistent profiles. Additionally, clinical and academic research protocols are meticulously optimized to achieve the best possible outcomes, with participants spending extended time in scanners to ensure high signal-to-noise ratio (SNR), and scanners across sites usually being from the same vendor. This contrasts with the situation in a network of diverse clinics, where acquisition protocols, clinical practices, and hardware vary significantly.

Currently, the predominant technique for dMRI harmonization is ComBAT (from the Dana-Farber Cancer Institute, Boston, Massachusetts). Unlike other approaches that harmonize the raw diffusion signal, ComBAT usually focuses on harmonizing dMRI scalar maps, such as Fractional Anisotropy (FA) and Mean Diffusivity (MD) maps, as well as other features such as cortical thickness and gray matter regions. ComBAT employs a Bayesian framework to learn the additive and multiplicative biases of each site, coupled with a linear model that captures the effects of covariates.

FIG. 9 presents a drawing illustrating an existing ComBAT technique. While existing ComBAT techniques are often effective in clinical trials and academic research settings, they may not be directly applicable to everyday clinical practice. Notably, as shown in FIG. 9A, existing ComBAT techniques typically harmonize all moving sites simultaneously, an approach that suits a fixed number of sites, but often falls short in a dynamic clinic network where the number of participants and acquisition sites expand over time. Additionally, existing ComBAT techniques presuppose a linear distribution of data with respect to its covariates, which is often not the case.

### SUMMARY

A computer system that performs pairwise harmonization is described. This computer may include: a computation device (such as a processor, a graphics processing unit or GPU, etc.) that executes program instructions; and memory that stores the program instructions. During operation, the computer system receives information specifying magnetic-resonance diffusion measurements, where the magnetic-resonance diffusion measurements are associated with at least an individual and a first site. Then, the computer system performs the pairwise harmonizing of the magnetic-resonance diffusion measurements based at least in part on target magnetic-resonance diffusion measurements, where the target magnetic-resonance diffusion measurements are associated with a population and one or more target sites, and the magnetic-resonance diffusion measurements and the target magnetic-resonance diffusion measurements acquired using different magnetic-resonance scanners. Next, the computer system performs quality control on the pairwise harmonized magnetic-resonance diffusion measurements, where the quality control includes comparing the pairwise harmonized magnetic-resonance diffusion measurements associated with the first site and the one or more target sites.

Note that the magnetic-resonance diffusion measurements may include measurements associated with the individual and a second individual.

Moreover, the magnetic-resonance diffusion measurements may be associated with a different number of individuals than the target magnetic-resonance diffusion measurements.

Furthermore, the quality control may include detecting a harmonization error in the pairwise harmonized magnetic-resonance diffusion measurements based at least in part on the pairwise harmonized magnetic-resonance diffusion measurements and a quality-control metric. The detecting may include segmenting the pairwise harmonized magnetic-resonance diffusion measurements into a first type of magnetic-resonance diffusion result or a second type of magnetic-resonance diffusion result.

Additionally, the performing of the pairwise harmonization may include eliminating one or more outliers in the magnetic-resonance diffusion measurements based at least in part on the pairwise harmonized magnetic-resonance diffusion measurements.

In some embodiments, the magnetic-resonance diffusion measurements may include tractography results. Moreover, the magnetic-resonance diffusion measurements may include: fractional anisotropy, apparent fiber density, an amount of free water, a demyelination metric, information associated with one or more brain regions (such as the cortical thickness, white and grey matter regions, one or more white and grey matter shape metrics, and/or the ventricles), scalar maps and/or scalar values.

Note that the pairwise harmonizing may reduce or eliminate additive and multiplicative bias in the magnetic-resonance diffusion measurements.

Furthermore, after performing the pairwise harmonization, the computer system may pool the magnetic-resonance diffusion measurements and the target magnetic-resonance diffusion measurements.

Additionally, after performing the pairwise harmonization, the computer system may generate a normative reference based at least in part on the magnetic-resonance diffusion measurements and the target magnetic-resonance diffusion measurements.

In some embodiments, the magnetic-resonance diffusion measurements may be associated with clinical operation of a magnetic-resonance scanner.

Note that the pairwise harmonization may facilitate personalized medicine for at least the individual.

Moreover, the performing of the pairwise harmonization may include: fitting the magnetic-resonance diffusion measurements using a data model; removing covariates and an intercept from the magnetic-resonance diffusion measurements using the data model; after removing the covariates and the intercept, computing variance (and/or bias) in the magnetic-resonance diffusion measurements; after computing the variance, correcting the magnetic-resonance diffusion measurements for multiplicative bias; and adding second covariates and a second intercept associated with the target magnetic-resonance diffusion measurements to the magnetic-resonance diffusion measurements. Note that a first data model for the target population (or site) may be fitted using maximum likelihood, while a second data model for the moving population (or site) may be fitted using maximum a posteriori (MAP). The prior for the MAP may be based at least in part on the first data model for the target population. More generally, data from the target population may be used as a prior for the computation of the parameters for the moving population. In some embodiments, the target population and the moving population may have their own sets of parameters.

Furthermore, the data model may include a nonlinear model. For example, the variance of the target population may be computed using maximum likelihood, while the variance of the moving population may be computed using MAP. Note that the prior term of the MAP may be based at least in part on the variance of the target population. Alternatively or additionally, the performing of the pairwise harmonization may include hyper-parameter autotuning with the target magnetic-resonance diffusion measurements to prevent overfitting to the data model.

Additionally, the performing of the pairwise harmonization may include supplementing the magnetic-resonance diffusion measurements with additional magnetic-resonance diffusion measurements from a different magnetic-resonance scanner when a number of individuals associated with the magnetic-resonance diffusion measurements is less than a predefined value. Note that the magnetic-resonance diffusion measurements and the additional magnetic-resonance diffusion measurements may have characteristics within a predefined value of each other.

Another embodiment provides a computer for use, e.g., in the computer system.

Another embodiment provides a computer-readable storage medium for use with the computer or the computer system. When executed by the computer or the computer system, this computer-readable storage medium causes the computer or the computer system to perform at least some of the aforementioned operations.

Another embodiment provides a method, which may be performed by the computer or the computer system. This method includes at least some of the aforementioned operations.

This Summary is provided for purposes of illustrating some exemplary embodiments, so as to provide a basic understanding of some aspects of the subject matter described herein. Accordingly, it will be appreciated that the above-described features are examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE FIGURES

FIG. 1 is a drawing of an example of an end-to-end tractometry/tractography pipeline in accordance with an embodiment of the present disclosure.

FIG. 2 is a block diagram illustrating an example of a computer system in accordance with an embodiment of the present disclosure.

FIG. 3 is a flow diagram illustrating an example of a method for performing pairwise harmonization using a computer system in FIG. 2 in accordance with an embodiment of the present disclosure.

FIG. 4 is a drawing illustrating an example of communication between components in a computer system in FIG. 2 in accordance with an embodiment of the present disclosure.

FIG. 5 is a drawing of an example of raw diffusion MRI (dMRI) in accordance with an embodiment of the present disclosure.

FIG. 6 is a drawing of an example of fractional anisotropy in accordance with an embodiment of the present disclosure.

FIG. 7 is a drawing of an example of unharmonized apparent fiber density in accordance with an embodiment of the present disclosure.

FIG. 8 is a drawing of an example of harmonized apparent fiber density in accordance with an embodiment of the present disclosure.

FIG. 9 is a drawing of examples of a Vanilla (existing) ComBAT technique and a Clinical ComBAT technique in accordance with an embodiment of the present disclosure.

FIG. 10 is a drawing of an example of a Vanilla ComBAT technique in accordance with an embodiment of the present disclosure.

FIG. 11 is a drawing of an example of harmonization of mean diffusivity using a Vanilla ComBAT technique in accordance with an embodiment of the present disclosure.

FIG. 12 is a drawing of an example of harmonization of mean diffusivity using a Vanilla ComBAT technique in accordance with an embodiment of the present disclosure.

FIG. 13 is a drawing of an example of harmonization using a Clinical ComBAT technique in accordance with an embodiment of the present disclosure.

FIG. 14 is a drawing of an example of outlier rejection in accordance with an embodiment of the present disclosure.

FIG. 15 is a drawing of examples of different polynomial models in accordance with an embodiment of the present disclosure.

FIG. 16 is a drawing of examples of harmonization of mean diffusivity using a Vanilla ComBAT technique and a Clinical ComBAT technique in accordance with an embodiment of the present disclosure.

FIG. 17 is a drawing of examples of harmonization of apparent fiber density using a Vanilla ComBAT technique and a Clinical ComBAT technique in accordance with an embodiment of the present disclosure.

FIG. 18 is a drawing of an example of harmonization using a Clinical ComBAT technique in accordance with an embodiment of the present disclosure.

FIG. 19 is a drawing of an example of a goodness of fit of the harmonization of mean diffusivity using a Clinical ComBAT technique in accordance with an embodiment of the present disclosure.

FIG. 20 is a drawing of an example of a hyperparameter tuning in accordance with an embodiment of the present disclosure.

FIG. 21 is a drawing of an example of a fit of mean diffusivity using a Clinical ComBAT technique in accordance with an embodiment of the present disclosure.

FIG. 22 is a drawing of an example of outlier rejection in accordance with an embodiment of the present disclosure.

FIG. 23 is a block diagram illustrating an example of a computer in accordance with an embodiment of the present disclosure.

Note that like reference numerals refer to corresponding parts throughout the drawings. Moreover, multiple instances of the same part are designated by a common prefix separated from an instance number by a dash.

### DETAILED DESCRIPTION

A computer system that performs pairwise harmonization is described. This computer may include: a computation device (such as a processor, a graphics processing unit or GPU, etc.) that executes program instructions; and memory that stores the program instructions. During operation, the computer system may receive information specifying magnetic-resonance diffusion measurements, where the magnetic-resonance diffusion measurements are associated with at least an individual and a first site. Then, the computer system may perform the pairwise harmonizing of the magnetic-resonance diffusion measurements based at least in part on target magnetic-resonance diffusion measurements, where the target magnetic-resonance diffusion measurements are associated with a population and one or more target sites, and the magnetic-resonance diffusion measurements and the target magnetic-resonance diffusion measurements may be acquired using different magnetic-resonance scanners. Next, the computer system may perform quality control on the pairwise harmonized magnetic-resonance diffusion measurements, where the quality control may include comparing the pairwise harmonized magnetic-resonance diffusion measurements associated with the first site and the one or more target sites.

By performing the pairwise harmonization, these Clinical ComBAT techniques may address the problems associated with existing harmonization techniques (which are sometimes referred to as ‘Vanilla ComBAT’). These capabilities may allow clinical data to be appropriately normalized and corrected for inter-site variations (e.g., in hardware, software, patient populations, etc.). These capabilities may allow clinical data to corrected as it is acquired and to be used to facilitate improved diagnosis of diseases and personalized treatments.

In the discussion that follows, the Clinical ComBAT techniques are used to analyze dMRI data associated with different MRI scanners at different sites or locations. However, the Clinical ComBAT techniques may be used to analyze a wide variety of types of magnetic-resonance images (which may or may not involve MRI, e.g., free-induction-decay measurements), such as: magnetic resonance spectroscopy (MRS) with one or more types of nuclei, magnetic resonance spectral imaging (MRSI), magnetic resonance elastography (MRE), magnetic resonance thermometry (MRT), magnetic-field relaxometry and/or another magnetic resonance technique (e.g., functional MRI, metabolic imaging, molecular imaging, blood-flow imaging, diffusion-tensor imaging, etc.). More generally (and provided that the result neurological fibers are in the same reference as the analyzed images), the Clinical ComBAT techniques may be used to analyze measurement results from a wide variety of invasive and non-invasive imaging techniques, such as: X-ray measurements (such as X-ray imaging, X-ray diffraction or computed tomography at one or more wavelengths between 0.01 and 10 nm), neutron measurements (neutron diffraction), electron measurements (such as electron microscopy or electron spin resonance), optical measurements (such as optical imaging or optical spectroscopy that determines a complex index of refraction at one or more visible wavelengths between 300 and 800 nm or ultraviolet wavelengths between 10 and 400 nm), infrared measurements (such as infrared imaging or infrared spectroscopy that determines a complex index of refraction at one or more wavelengths between 700 nm and 1 mm), ultrasound measurements (such as ultrasound imaging in an ultrasound band of wavelengths between 0.2 and 1.9 mm), proton measurements (such as proton scattering), positron emission spectroscopy, positron emission tomography (PET), impedance measurements (such as electrical impedance at DC and/or an AC frequency) and/or susceptibility measurements (such as magnetic susceptibility at DC and/or an AC frequency).

We now describe embodiments of the Clinical ComBAT techniques. The brain is composed of three main regions: the gray matter (GM), the white matter (WM) and cerebrospinal fluid (CSF). The white matter is composed of axons and glial cells (such as astrocytes, microglias, oligodendrocytes, etc.), which are surrounded by water. An axon is a myelinated and elongated portion of a nerve cell that conducts electric pulses between cortical regions. Axons are organized in fibers, which are themselves grouped into bundles that one can picture as large brain connection pathways. Note that the myelin sheaths are wrapped around axons. Moreover, the glial cells include oligodendrocyte, astrocyte and microglia. All the white-matter components are surrounded by cerebrospinal fluid.

With recent advances in imaging technologies, it is possible to measure the connectivity of the brain and quantify white-matter microstructure, as well as its deterioration, typically due to aging or some neurodegenerative disease. Thanks to the fibrous nature of axons, the diffusion of molecules in the white matter is anisotropic and can be measured by a non-invasive technology, such as dMRI.

Diffusion imaging is the process by which the average displacement of water molecules within a voxel (or volume element, which is sometimes referred to as a ‘voxel’) is measured. A dMRI acquisition protocol is typically designed to acquire a series of diffusion weighted images that are each sensitized to the water diffusion into a particular direction. These images are usually acquired following the seminal pulse gradient spin echo protocol (or a slight variation of it). In an MRI scanner, a strong permanent magnetic field combined with the interplay of radio-frequency pulses and diffusion-sensitizing gradients (which are referred to as ‘pulsed field gradients’ or PFG).

Given the pulse duration and pulse separation, each PFG has a magnitude or strength (which is typically called b-value) and a unique pre-defined orientation (which is typically called b-vector). Mathematically, this amounts to G=∥G∥·u, where G is the gradient, ∥G∥ is the norm of that vector (the b-value) and u is a unit vector (the b-vector). An arbitrary large number of diffusion weighted images can be acquired, each with a different PFG b-value/b-vector pair.

When the diffusion weighted images were acquired with the same PFG b-value but different b-vectors, the PFGs can be illustrate as points lying on a sphere whose distance to the origin equals the PFG b-value. This configuration is called single-shell. However, when different b-values are used, the PFG points may lie on different concentric spheres in a configuration called multi-shell. While these q-space sampling techniques are often used, other q-space configurations may occur, such as: spectrum sampling, random sampling, grid sampling, and sparse sampling. Note that a dot at the center of the spheres indicates the b0 image, which amounts to a b-value of zero. The b0 image is T₂-weighted image that takes into account tissue contrasts and signals in the absence of diffusion gradients. The b0 serves as an anatomical baseline image and is often used to normalize diffusion weighted images.

Thus, diffusion images encode how water molecules diffuse along certain directions. Depending on the environment surrounding a water molecule (such as fat, muscle, fibrous tissues, free water, etc.) it may move more or less freely and, therefore, may return a more or less intense signal. By sampling the q-space with different PFG b-values and b-vectors, a voxel may get different signal values, which can be combined into a compact mathematical model that represents the underlying local tissue structure.

The accuracy of such a local model typically depends heavily on the total number of acquired diffusion-weighted images. The simplest model is one derived from a single diffusion acquisition (or one point in the q-space). This results in a raw diffusion-weighted image whose voxel's intensity is related to the level of sensitization along one direction and to the local tissue structure. While this simple acquisition can be used for clinical applications (thanks to its simplicity and the short time, e.g., a few minutes, it takes to acquire it), it often has limitations. Notably, given that the measured signal S after the PFG is given by

$$S = {S}_{0} {e}^{- bD} ,$$

where S₀ is the magnetic resonance (MR) signal at baseline, b is the b-value and D is the diffusion coefficient. Therefore, the acquired image may include the raw signal S, but not the diffusion coefficient, per se. Also, because the term e^{-bD} typically behaves very much like the T₂-weighting term e^{-TE/T2} (where TE is the echo time and T₂ the transverse relaxation time), the measured value S is usually heavily affected by relaxation times. This results in variations in the image intensities that can be hard to interpret. In addition, a diffusion image usually has a low signal-to-noise ratio (SNR), which makes it difficult to distinguish the underlying anatomy.

In order to compensate for the T₂ contamination and the low SNR, more diffusion-weighted images need to be acquired. Consequently, when two diffusion images with different b-values are acquired and combined, the T₂ contamination can be cancelled out and the diffusion coefficient D (which is often called the ‘apparent diffusion coefficient image’ or ADC image) can be recovered:

$$ADC = D = \frac{ln ( \frac{{S}_{2}}{{S}_{1}} )}{b 1 - b 2} ,$$

where S₁ and S₂ are signal intensities obtained with b1 and b2 b-values. Note that when one of the two signals is obtained with a b0, the following ADC map (which is also called the attenuation map) is obtained:

$$ADC = \frac{ln ( \frac{{S}_{0}}{{S}_{1}} )}{b 1} . \qquad ( 1 )$$

ADC maps are widely used and have been shown to help diagnose brain strokes.

Moreover, in order to increase the SNR, three diffusion images may be acquired. A typical approach is to use the same b-value for all three images, but with b-vectors aligned on the X, Y and Z canonical axis:

$${S}_{x} = {S}_{o} {e}^{- {bD}_{xx}} ,$$

$${S}_{y} = {S}_{o} {e}^{- {bD}_{yy}} , and$$

$${S}_{z} = {S}_{o} {e}^{- {bD}_{zz}} ,$$

where Dₓₓ, D_{yy}, D_{zz} are the diffusion coefficients along the three axes. The combination of these images results in the so-called trace DW image (which is sometimes referred to as the short DWI). The combination may be implemented as follows:

$${S}_{DWI} = \sqrt[3]{{S}_{x} {S}_{y} {S}_{z}} = {S}_{o} {e}^{- b ( {D}_{xx} + {D}_{yy} + {D}_{zz} ) / 3} = {S}_{0} {e}^{- b ( {D}_{trace ) / 3}} . \qquad ( 2 )$$

Note that D_{trace} is the trace of the diffusion tensor.

Compared to a single diffusion image S, S_{DWI} typically offers significantly improved contrast. In order to remove the T₂ contamination, a b0 image may be acquired and an attenuated ADC map may be computed following Eqn. 1:

$$ADC = \frac{ln ( \frac{{S}_{0}}{{S}_{DWJ}} )}{b} = \frac{{D}_{trace}}{3} . \qquad ( 3 )$$

Note that, by their very nature, the signal intensities of an ADC map are opposite to those of a DWI, which can be confusing. In order to see this, contemplate from Eqn. 2 that S_{DWI} is maximum when D_{trace} is 0 and decreases exponentially fast as D_{trace} is increasingly positive. This is the exact opposite with the ADC map of Eqn. 3, which is minimum when D_{trace} is 0. Thus, tissues with large diffusivity values (such as free water and cerebrospinal fluid) usually appear bright in an ADC map, while tissues with low diffusivity values (such as stroke and fibrous tissues) typically appear dark. These contrasts are the exact opposite in a DWI image. In order to alleviate the confusion, an exponential ADC image (which is sometimes referred to as a Stejskal-Tanner attenuation map) may be computed:

$${S}_{ex p} = \frac{{S}_{DWI}}{{S}_{0}} = {e}^{- b . ADC} . \qquad ( 4 )$$

By acquiring more diffusion images, the 3D structure of the brain tissue may be measured. As discussed previously, the fibrous nature of axons makes the white matter a highly anisotropic environment, with diffusion coefficients larger along the axonal tracts than perpendicular to the tracts. One way of recovering the 3D microstructure of the white matter is by acquiring at least seven images, such as six diffusion-weighted images from six different b-vectors (S₁ to S₆) plus a b0 image (S₀) to recover the unimodal model that is referred to as the diffusion tensor. The diffusion tensor is a 3×3 symmetric positive definite matrix:

$$( \begin{matrix}{D}_{xx} & {D}_{xy} & {D}_{xz} \\ {D}_{yx} & {D}_{yy} & {D}_{yz} \\ {D}_{zx} & {D}_{zy} & {D}_{zz}\end{matrix} ) . \qquad ( 5 )$$

From this matrix, the ADC can be represented as D(u)=u^{T}Du, where u is a b-vector and the b0 attenuated signal is

$${S}_{u} = {e}^{- b . ( {u}^{T} Du )} .$$

Because D has six independent variables, it can be recovered from six (or more) diffusion-weighted images using least-square regression, weighted least-square regression or another technique involving Rician noise.

Moreover, from the diffusion tensor D, one can extract three eigenvalues (λ₁, λ₂ and λ₃) whose combination leads to diffusion metrics, such as the fractional anisotropy (FA), the mean diffusivity (MD), the radial diffusivity (RD) and the axial diffusivity (AD), where λ₁, λ₂ and λ₃ are the eigenvalues of the diffusion tensor. Because each voxel is associated with a diffusion tensor, note that these diffusion metrics can be visualized as image modalities. Fractional anisotropy is often useful for the assessment of children's brain maturation, dyslexia, and for some psychiatric disorders.

One challenge associated with the diffusion tensor D is that it only encodes one white matter fiber direction. Considering that more than two-thirds of the white matter voxels contain crossing brain fibers oriented in more than one direction, this model is often regarded as ill-suited for many applications. As a solution, multi-directional local models have been proposed, such as the bi-tensor and multi-tensor models. Other models account for higher orders by using spherical deconvolution (SD) approaches. In these cases, instead of assuming a fixed number of local fiber orientations, the SD models approximate the local white-matter structure using so-called ODF models or fiber ODF. Note that the SD approaches express the acquired diffusion signal in each voxel as a spherical convolution between an ODF and a low-pass filter called a fiber response function (FRF) that describes the signal profile of the white matter. Deconvoluting the measured signal with a FRF leads to the desired fODF.

Tractography is a process in which fibers are reconstructed by iteratively following the local fiber orientation models. When a local fiber orientation is put on each white matter voxel, the underlying fibrous structure of the white matter tissues may be determined. Moreover, with these models, several tractography techniques have been proposed to reconstruct brain fibers. When enough fibers have been reconstructed, so that each voxel is traversed by at least one fiber, one obtains a so-called whole brain tractogram (which may contain millions of fibers).

While some global tractography techniques have been proposed, typical approaches involve a local iterative technique that builds fibers point by point. The local tractography techniques can be deterministic or probabilistic, may use particle filtering (such as a Particle Filtering Tractography or PFT technique), may be driven by a pre-computed cortex manifold (such as a Surface Enhanced Tracking or SET technique), and/or may be specific to pre-segmented white matter regions (such as the Bundle Specific Tracking or BST technique).

In general, all tracking techniques have limitations, because they all produce spurious or anatomically implausible brain fibers. Such fibers may be too short or too long, have a loopy shape, pass-through non-white matter regions, connect inappropriate gray matter regions and/or connect no gray matter regions at all. These fibers are typically filtered out in a post-processing operation. The remaining anatomically-plausible fibers may then be grouped into fiber bundles.

Fibers in a bundle have roughly the same shape and connect the same gray matter regions. The most obvious way to extract white-matter bundles from a whole brain tractogram is by asking a neuroanatomist to manually dissect bundles of interest. However, manual dissecting bundles is long, tedious, and prone to large inter- and intra-expert variability. Automated techniques, such as QuickBundles (QB), QuickBundlesX (QBx), Deep Fiber Clustering (DFC), RecoBundles (RB), RecoBundlesX (RBx), TractSeg, XTRACT, FINTA, FIESTA, Deep White Matter Analysis (WMA), and BINTA, have been proposed to accelerate and increase the reproducibility of this process.

Bundles can be useful to assess the integrity of the white matter into specific regions associated with some neurodegenerative diseases, such as Alzheimer's disease. White-matter bundles open the door to tractometry analysis, in which white-matter metrics (such as the fractional anisotropy, axial diffusivity, radial diffusivity, and mean diffusivity) are analyzed within each bundle. These metrics can be analyzed across the entire bundles or within sub-regions. Note that more advanced metrics based at least in part on high-dimensional local models (be they single- or multi-shell) may also be computed, such as: free-water indices, myelin water fraction, neurite density index, orientation dispersion index, and/or magnetization transfer ratio.

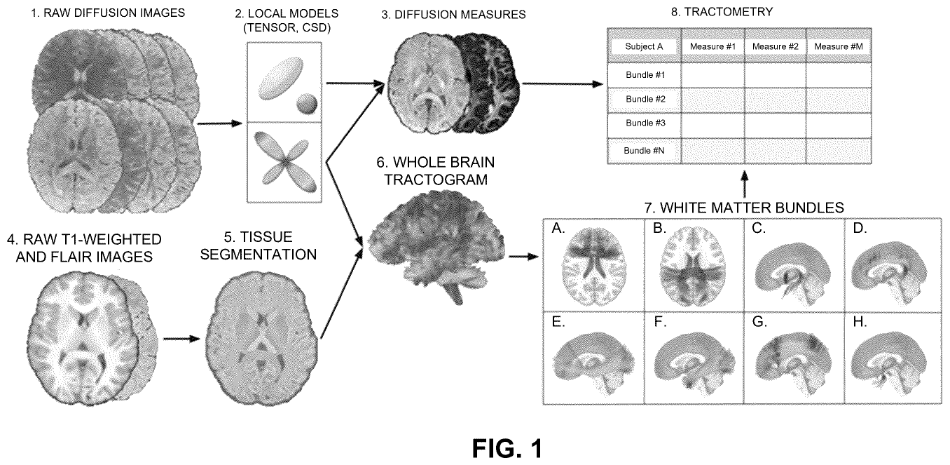

Tractography is a well-established technique for brain connectivity analysis, and has been shown to be effective in measuring the local brain microstructure. While tractography and tractometry can be used independently, they are often used jointly. FIG. 1 presents a drawing of an example of an end-to-end tractometry/tractography pipeline for converting raw input diffusion and structural images into a tractometry report.

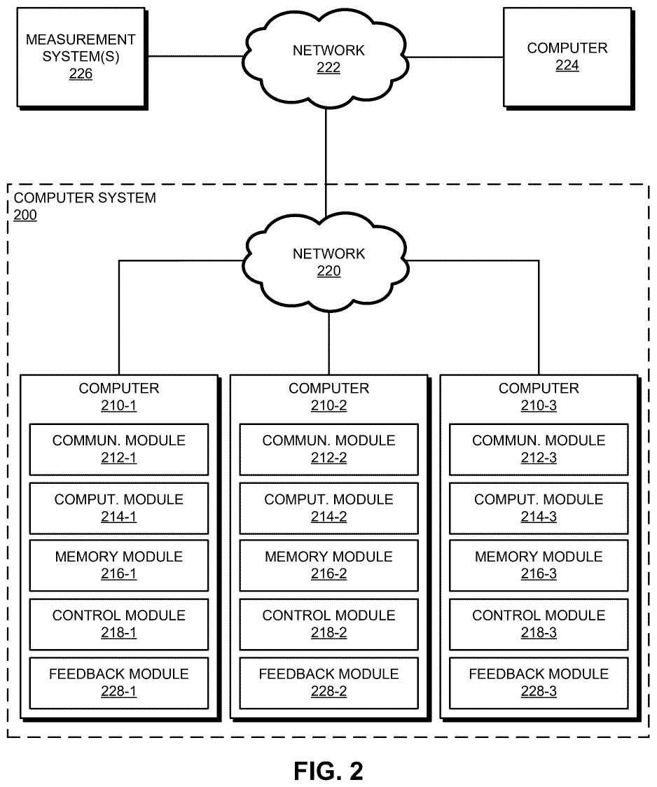

We now describe a computer system that may implement the Clinical ComBAT techniques. FIG. 2 presents a block diagram illustrating an example of a computer system 200. This computer system may include one or more computers 210. These computers may include: communication modules 212, computation modules 214, memory modules 216, and optional control modules 218. Note that a given module or engine may be implemented in hardware and/or in software.

Communication modules 212 may communicate frames or packets with data or information (such as measurement results or control instructions) between computers 210 via a network 220 (such as the Internet and/or an intranet). For example, this communication may use a wired communication protocol, such as an Institute of Electrical and Electronics Engineers (IEEE) 802.3 standard (which is sometimes referred to as ‘Ethernet’) and/or another type of wired interface. Alternatively or additionally, communication modules 212 may communicate the data or the information using a wireless communication protocol, such as: an IEEE 802.11 standard (which is sometimes referred to as ‘Wi-Fi’, from the Wi-Fi Alliance of Austin, Texas), Bluetooth (from the Bluetooth Special Interest Group of Kirkland, Washington), a third generation or 3G communication protocol, a fourth generation or 4G communication protocol, e.g., Long Term Evolution or LTE (from the 3rd Generation Partnership Project of Sophia Antipolis, Valbonne, France), LTE Advanced (LTE-A), a fifth generation or 5G communication protocol, other present or future developed advanced cellular communication protocol, or another type of wireless interface. For example, an IEEE 802.11 standard may include one or more of: IEEE 802.11a, IEEE 802.11b, IEEE 802.11g, IEEE 802.11-2007, IEEE 802.11n, IEEE 802.11-2012, IEEE 802.11-2016, IEEE 802.11ac, IEEE 802.11ax, IEEE 802.11ba, IEEE 802.11be, IEEE 802.11bn or other present or future developed IEEE 802.11 technologies.

In the described embodiments, processing a packet or a frame in a given one of computers 210 (such as computer 210-1) may include: receiving the signals with a packet or the frame; decoding/extracting the packet or the frame from the received signals to acquire the packet or the frame; and processing the packet or the frame to determine information contained in the payload of the packet or the frame. Note that the communication in FIG. 2 may be characterized by a variety of performance metrics, such as: a data rate for successful communication (which is sometimes referred to as ‘throughput’), an error rate (such as a retry or resend rate), a mean squared error of equalized signals relative to an equalization target, intersymbol interference, multipath interference, a signal-to-noise ratio, a width of an eye pattern, a ratio of number of bytes successfully communicated during a time interval (such as 1-10 s) to an estimated maximum number of bytes that can be communicated in the time interval (the latter of which is sometimes referred to as the ‘capacity’ of a communication channel or link), and/or a ratio of an actual data rate to an estimated data rate (which is sometimes referred to as ‘utilization’). Note that wireless communication between components in FIG. 2 uses one or more bands of frequencies, such as: 900 MHz, 2.4 GHZ, 5 GHZ, 6 GHZ, 60 GHZ, the Citizens Broadband Radio Spectrum or CBRS (e.g., a frequency band near 3.5 GHz), and/or a band of frequencies used by LTE or another cellular-telephone communication protocol or a data communication protocol. In some embodiments, the communication between the components may use multi-user transmission (such as orthogonal frequency division multiple access or OFDMA).

Moreover, computation modules 214 may perform calculations using: one or more microprocessors, ASICs, microcontrollers, programmable-logic devices, GPUs and/or one or more digital signal processors (DSPs). Note that a given computation component is sometimes referred to as a ‘computation device’.

Furthermore, memory modules 216 may access stored data or information in memory that local in computer system 200 and/or that is remotely located from computer system 200. Notably, in some embodiments, one or more of memory modules 216 may access stored measurement results in the local memory, such as dMRI data for one or more individuals (which, for multiple individuals, may include cases and controls or disease and healthy populations). Alternatively or additionally, in other embodiments, one or more memory modules 216 may access, via one or more of communication modules 212, stored measurement results in the remote memory in computer 224, e.g., via network 220 and network 222. Note that network 222 may include: the Internet and/or an intranet. In some embodiments, the measurement results are received from one or more measurement systems 226 (such as MRI scanners) via network 220 and network 222 and one or more of communication modules 212. Thus, in some embodiments at least some of the measurement results may have been received previously and may be stored in memory, while in other embodiments at least some of the measurement results may be received in real-time from the one or more measurement systems 226.

While FIG. 2 illustrates computer system 200 at a particular location, in other embodiments at least a portion of computer system 200 is implemented at more than one location. Thus, in some embodiments, computer system 200 is implemented in a centralized manner, while in other embodiments at least a portion of computer system 200 is implemented in a distributed manner. For example, in some embodiments, the one or more measurement systems 226 may include local hardware and/or software that performs at least some of the operations in the Clinical ComBAT techniques. This remote processing may reduce the amount of data that is communicated via network 220 and network 222. In addition, the remote processing may anonymize the measurement results that are communicated to and analyzed by computer system 200. This capability may help ensure computer system 200 is compatible and compliant with regulations, such as the Health Insurance Portability and Accountability Act, e.g., by removing or obfuscating protected health information in the measurement results.

Although we describe the computation environment shown in FIG. 2 as an example, in alternative embodiments, different numbers or types of components may be present in computer system 200. For example, some embodiments may include more or fewer components, a different component, and/or components may be combined into a single component, and/or a single component may be divided into two or more components.

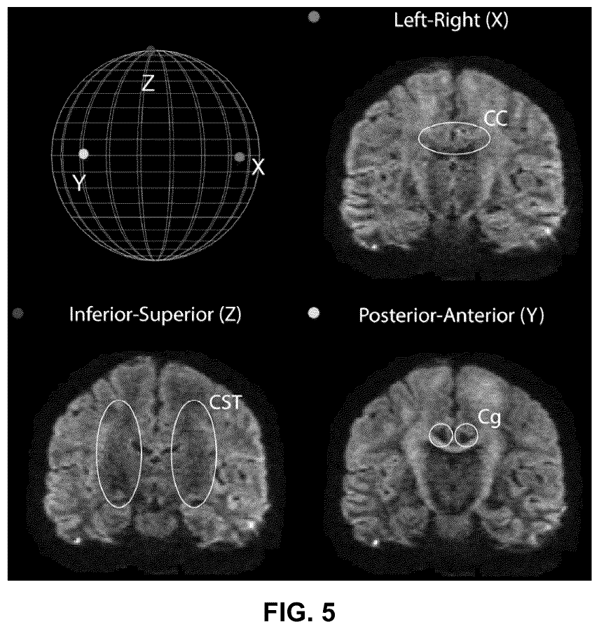

As discussed previously, existing ComBAT techniques often suffer from a number of problems. Moreover, as described further below with reference to FIGS. 5-22, in order to address these challenges computer system 200 may perform the Clinical ComBAT techniques. Notably, during the Clinical ComBAT techniques, one or more of optional control modules 218 may divide the analysis among computers 210. Then, a given computer (such as computer 210-1) may perform at least a designated portion of the analysis. In particular, computation module 214-1 may receive (e.g., access) information (e.g., using memory module 216-1) specifying medical-imaging data that specify the central nervous system (including white matter) for one or more individuals. Note that the medical-imaging data may include or may correspond to dMRI data. Then, computation module 214-1 may perform operations in an analysis pipeline and/or a Clinical ComBAT technique.

For example, the analysis pipeline may include: QC (such as visual inspection, an image resolution check and/or a gradient distribution check), skull stripping or SS, preprocessing or PP (such as denoising, motion correction and/or a correction for magnetic field inhomogeneity), harmonization (to correct for data variation, e.g., in dMRI and/or MRI data, associated with different MR scanners), tissue segmentation (e.g., white matter, grey matter, tumor, cerebrospinal fluid, etc., and which may be based at least in part on structural MRI data using a convolutional neural network). The output of these portions or stages of the analysis pipeline may include tractography results (which are sometimes referred to as ‘tractograms’) that specify a set of neurological fibers, which are sometimes referred to as ‘neural tracts’ or ‘streamlines’. In the present discussion, note that a ‘neurological fiber’ includes a sequence of three dimensional or 3D points that are connected together.

Moreover, computation module 214-1 may perform additional tractography by computing, using a predetermined (e.g., pretrained) autoencoder neural network, second tractography results that specify a second set of neurological fibers based at least in part on the tractography results and information associated with a neurological anatomical region. Note that a subset of the set of neurological fibers may be anatomically implausible and the second set of fibers may exclude the subset. Moreover, the predetermined autoencoder neural network may be trained using an unsupervised-learning technique. In some embodiments, the second set of neurological fibers may, at least in part, be different from the set of neurological fibers. Note that computing the second tractography results may include a cleaning and bundling operation in which the neurological fibers are grouped or bundled into different types of bundles of neurological fibers having different numbers of neurological fibers.

Next, computation module 214-1 may perform additional operations in multiple stages in the analysis pipeline. For example, the second tractography results may be input to or used by one or more stages or operations in the analysis pipeline, such as: statistical analysis, connectome analysis, population analysis, etc. Notably, the analysis pipeline may include: microstructure analysis, region-wise microstructure statistics (RWMS), and/or region-wise statistical analysis (RWSA). In the case of analysis for an individual, region-wise statistical analysis may be based at least in part on a comparison with a reference atlas or data structure (such as a region-wise microstructure brain atlas corresponding to multiple individuals). Alternatively, in the case of analysis of a population, at least a portion of the analysis may be computed based at least in part on cases and controls in the population.

After performing the operations in the stages in the analysis pipeline, computation module 214-1 may output a subset of white-matter disease biomarkers for different neurological anatomical regions for at least the designated portion of the analysis. For a given neurological anatomical region, the set of white-matter disease biomarkers may include: an apparent fiber density, which corresponds to a total intra-axonal volume; an amount of free water; or a demyelination metric. Note that the amount of free water may correspond to neuroinflammation, and/or the apparent fiber density may correspond to axonal disruption or axonal quality. The apparent fiber density may be recovered or determined using: a diffusion fractional anisotropy, water corrected fractional anisotropy, water corrected axial diffusivity, intracellular neurite orientation dispersion and density imaging (NODDI) volume fraction, and/or a local fODF. Moreover, the demyelination metric may include: an inverse free-water-corrected radial diffusivity, a ratio of T₁ to T₂, a magnetization transfer ratio, or MWF. As noted previously, the MWF may be the ratio of the signal amplitudes measured in or associated with two water compartments

$$MWF = \frac{M W}{( M W + EICW )} ,$$

where MW is the signal amplitude associated with a myelin water compartment and EICW is a signal amplitude associated with an extra-intra cellular water compartment. These signal amplitude values may be obtained via a multi-exponential function and nonlinear optimization from a series of acquired T₂-weighted images at different echo times (e.g., up to 48 echos). In some embodiments, the set of white-matter disease biomarkers may be computed on a per-voxel basis and/or a per-neurological-fiber (or fixel) basis.

Furthermore, computation module 214-1 may perform one or more operations in a Clinical ComBAT technique to correct for data variation, e.g., in dMRI and/or MRI data, associated with different MR scanners. Embodiments of Clinical ComBAT techniques are described further below with reference to FIGS. 5-22.

Additionally, one or more feedback modules 228 in computer system 200 may use the set of white-matter disease biomarkers for different neurological anatomical regions to provide feedback information associated with at least the individual or the population. For example, the set of white-matter disease biomarkers for different neurological anatomical regions may be inputs to a pretrained predictive model (such as a supervised machine-learning model or a neural network), which outputs the feedback information. Note that the feedback information may include: diagnostic information, information associated with disease progression (such as a disease stage), information regarding efficacy of a treatment, or a treatment recommendation (e.g., based at least in part on the disease stage). In some embodiments, the feedback information (such as the diagnostic information) may be based at least in part on a volume of a neurological anatomical region in at least the individual, such as the hippocampus.

In these ways, computer system 200 may automatically and accurately analyze white matter for at least an individual or a population, such as: perform filtering and/or grouping or bundling of the neurological fibers, computing the set of white-matter disease biomarkers, etc. These capabilities may allow computer system 200 to perform subsequent analyses or one or more additional operations, such as: connectome analysis, white matter segmentation, one or more clinical trial enrollment or exclusion criteria, assessing the impact of a medical intervention for a disease (e.g., in a clinical trial for a candidate pharmaceutical agent, neurostimulation and/or another type of therapy), precision medicine (such as in selecting a correct medical intervention to treat a disease, e.g., as a companion diagnostic for a prescription drug or a dose of a prescription drug), etc. For example, the more-accurate white-matter results may allow more accurate determination of: an axon density index, radial diffusion, volume, a myelin index or metric, an inflammation index or metric, fractional anisotropy (which is sensitive to anomalies), free water, apparent fiber density, a microglia index or metric, an astrocyte index or metric, an oligodendrocyte index or metric, a lesion edema index or metric, a cerebrospinal fluid index or metric and/or, more generally, a characteristic or attribute associated with microstructure environment (e.g., on a sub-millimeter length scale that is less than a length scale corresponding to a voxel) of one or more neurological fibers (and, more generally, white matter) in particular neurological anatomic regions. Note that the disease may include: Parkinson's disease, Alzheimer's disease, multiple sclerosis, amyotrophic lateral sclerosis (ALS) or Lou Gehrig's disease, normal pressure hydrocephalus, concussion, migraine, epilepsy, a type of cancer, an autoimmune disease, schizophrenia, depression, bipolar disorder, another type of mental illness, traumatic brain injury, an alcohol-use disorder, a neurodegenerative disease, a neuroinflammatory disease, or another type of neurological disease of the central nervous system. Consequently, the Clinical ComBAT techniques may facilitate accurate, value-added use of the measurement results, such as medical-imaging data.

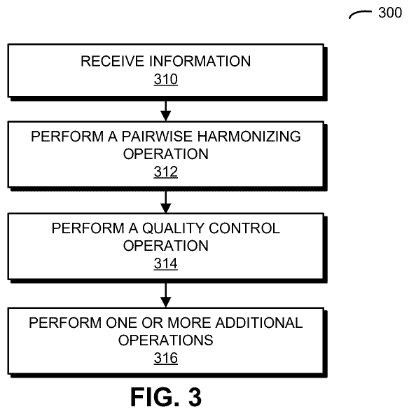

We now describe embodiments of the method. FIG. 3 presents a flow diagram illustrating an example of a method 300 for performing pairwise harmonization, which may be performed by a computer system (such as computer system 200 in FIG. 2). During operation, the computer system may receive information (operation 310) specifying magnetic-resonance diffusion measurements, where the magnetic-resonance diffusion measurements are associated with at least an individual and a first site. For example, the receiving may include accessing the information in memory. Note that the magnetic-resonance diffusion measurements may be associated with magnetic-resonance images of at least the individual, including: T₁-weighted, T₂-weighted, proton density, fluid-attenuated inversion recovery (FLAIR), b-value images, diffusion weighted images, diffusion tensor images, high angular resolution diffusion images, and/or another type of magnetic-resonance image, data or derived metric (such as the fractional anisotropy, the mean diffusivity, the apparent fiber density, etc.). In some embodiments, harmonization is also performed for brain region information (such as the size, length, shape, etc.).

Then, the computer system may perform the pairwise harmonizing (operation 312) of the magnetic-resonance diffusion measurements based at least in part on target magnetic-resonance diffusion measurements, where the target magnetic-resonance diffusion measurements are associated with a population and one or more target sites, and the magnetic-resonance diffusion measurements and the target magnetic-resonance diffusion measurements acquired using different magnetic-resonance scanners.

Next, the computer system may perform a quality control (operation 314) on the pairwise harmonized magnetic-resonance diffusion measurements, where the quality control includes comparing the pairwise harmonized magnetic-resonance diffusion measurements associated with the first site and the one or more target sites.

Note that the magnetic-resonance diffusion measurements may include measurements associated with the individual and a second individual.

Moreover, the magnetic-resonance diffusion measurements may be associated with a different number of individuals than the target magnetic-resonance diffusion measurements.

Furthermore, the quality control may include detecting a harmonization error in the pairwise harmonized magnetic-resonance diffusion measurements based at least in part on the pairwise harmonized magnetic-resonance diffusion measurements and a quality-control metric. The detecting may include segmenting the pairwise harmonized magnetic-resonance diffusion measurements into a first type of magnetic-resonance diffusion result or a second type of magnetic-resonance diffusion result.

Additionally, the magnetic-resonance diffusion measurements may include tractography results. Moreover, the magnetic-resonance diffusion measurements may include: fractional anisotropy, apparent fiber density, an amount of free water, and/or a demyelination metric.

Note that the pairwise harmonizing may reduce or eliminate additive and multiplicative bias in the magnetic-resonance diffusion measurements.

Moreover, the magnetic-resonance diffusion measurements may be associated with clinical operation of a magnetic-resonance scanner.

Furthermore, the pairwise harmonization may facilitate personalized medicine for at least the individual.

In some embodiments, the computer system may optionally perform one or more additional operations (operation 316). For example, the performing of the pairwise harmonization (operation 312) may include eliminating one or more outliers in the magnetic-resonance diffusion measurements based at least in part on the pairwise harmonized magnetic-resonance diffusion measurements.

Furthermore, after performing the pairwise harmonization (operation 312), the computer system may pool the magnetic-resonance diffusion measurements and the target magnetic-resonance diffusion measurements.

Additionally, after performing the pairwise harmonization (operation 312), the computer system may generate a normative reference based at least in part on the magnetic-resonance diffusion measurements and the target magnetic-resonance diffusion measurements.

Note that the performing of the pairwise harmonization (operation 312) may include: fitting the magnetic-resonance diffusion measurements using a data model; removing covariates and an intercept from the magnetic-resonance diffusion measurements using the data model; after removing the covariates and the intercept, computing variance in the magnetic-resonance diffusion measurements; after computing the variance, correcting the magnetic-resonance diffusion measurements for multiplicative bias; and adding second covariates and a second intercept associated with the target magnetic-resonance diffusion measurements to the magnetic-resonance diffusion measurements.

Moreover, the data model may include a nonlinear model. For example, the variance may be computed using maximum likelihood for the target population and MAP for the moving population. Alternatively or additionally, the performing of the pairwise harmonization may include hyper-parameter autotuning with the target magnetic-resonance diffusion measurements to prevent overfitting to the data model.

Furthermore, the performing of the pairwise harmonization (operation 312) may include supplementing the magnetic-resonance diffusion measurements with additional magnetic-resonance diffusion measurements from a different magnetic-resonance scanner when a number of individuals associated with the magnetic-resonance diffusion measurements is less than a predefined value. Note that the magnetic-resonance diffusion measurements and the additional magnetic-resonance diffusion measurements may have characteristics within a predefined value of each other.

In some embodiments of method 300, there may be additional or fewer operations. Furthermore, the order of the operations may be changed, and/or two or more operations may be combined into a single operation.

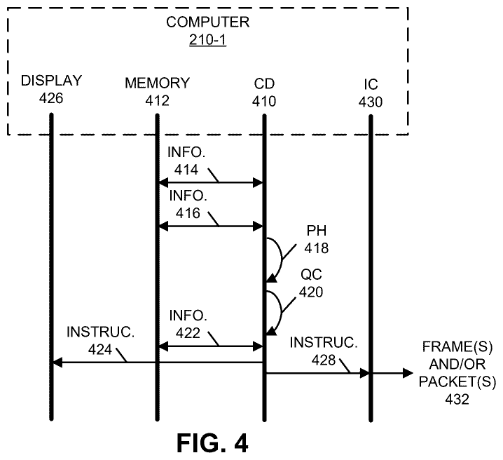

Embodiments of the Clinical ComBAT techniques are further illustrated in FIG. 4, which presents a drawing illustrating an example of communication among components in computer system 200. In FIG. 4, a computation device (CD) 410 (such as a processor or a GPU) in computer 210-1 may access, in memory 412 in computer 210-1, information 414 specifying dMRI measurements for at least an individual and a first site. After receiving dMRI measurements, computation device 410 may may access, in memory 412 in computer 210-1, information 416 specifying target dMRI measurements.

Then, computation device 410 may perform pairwise harmonizing (PH) 418 of the dMRI measurements based at least in part on target dMRI measurements, where the target dMRI measurements are associated with a population and one or more target sites, and the dMRI measurements and the target dMRI measurements acquired using different magnetic-resonance scanners.

Next, computation device 410 may perform quality control (QC) 420 on the pairwise harmonized dMRI measurements, where quality control 420 includes comparing the pairwise harmonized dMRI measurements associated with the first site and the one or more target sites.

In some embodiments, computation device 410 may store, in memory 412, information 422 specifying the pairwise harmonized dMRI measurements and/or results of quality control 420 on the pairwise harmonized dMRI measurements. Alternatively or additionally, computation device 410 may provide instructions 424 to display information 422 on display 426 in computer 210-1. In some embodiments, computation device 410 may provide instructions 428 to interface circuit 430 in computer 210-1 to transmit, to another electronic device, one or more frames or packets 432 that include information 422.

While FIG. 4 illustrates communication between components using unidirectional or bidirectional communication with lines having single arrows or double arrows, in general the communication in a given operation in this figure may involve unidirectional or bidirectional communication.

Moreover, while the preceding discussion illustrated the Clinical ComBAT techniques with data from different magnetic-resonance scanners, in other embodiments, the diffusion magnetic-resonance measurements and/or the target diffusion magnetic-resonance measurements may come or may be acquired using the same physical magnetic-resonance scanner, but at different time points (which may result in signal drift), after a software update, using a different acquisition protocol, and/or for different populations.

In some embodiments, the computer may provide feedback information associated with at least the individual based at least in part on a set of white-matter disease biomarkers in different neurological anatomical regions. Moreover, the feedback information may include: diagnostic information, information associated with disease progression (such as a disease stage), information regarding efficacy of a treatment, or a treatment recommendation (e.g., based at least in part on the disease stage). For example, the diagnostic information may be associated with a neurological or neurodegenerative disease, including: epilepsy, depression, autism, cerebral palsy, multiple sclerosis, Alzheimer's disease, Parkinson's disease, amyotrophic lateral sclerosis, traumatic brain injury, an alcohol-use disorder, or another neurological or neurodegenerative disease. Furthermore, the feedback information may be based at least in part on a volume of a neurological anatomical region in at least the individual, such as the hippocampus.

Moreover, the medical-imaging data may be associated with a population of cases and controls, which includes at least the individual, and the feedback information may be associated with the population.

Furthermore, the Clinical ComBAT and/or the feedback information may be determined using a pretrained predictive model. For example, the pretrained predictive model may include: a machine-learning model or a neural network. Note that a given node in a given layer in the neural network may include an activation function, and the activation function may include: a rectifier (ReLU), a leaky ReLU, an exponential linear unit (ELU) activation function, a parametric ReLU, a tanh activation function, or a sigmoid activation function.

Additionally, the set of white-matter disease biomarkers includes free-water, apparent-fiber-density and myelin metrics. These white matter disease biomarkers may be used in conjunction with different neurological diseases or neurodegenerative diseases. For example, the neurological diseases may include: Alzheimer's disease, Parkinson's disease, multiple sclerosis, traumatic brain injury, depression, amyotrophic lateral sclerosis and/or chronic traumatic encephalopathy.

In some embodiments, the set of white-matter disease biomarkers may be associated with Alzheimer's disease, Parkinson's disease and/or multiple sclerosis. Because Alzheimer's disease, Parkinson's disease and multiple sclerosis are, in general, slowly progressing neurological diseases, they often have corresponding signatures associated with them. Notably, in their early stages, Alzheimer's disease, Parkinson's disease and multiple sclerosis are characterized by inflammation of the white matter, which typically causes the free-water metric to increase. At this stage, the diseases are often unknown to the patients and the apparent-fiber-density and myelin metrics are usually stable. However, in later disease stages, the apparent-fiber-density and myelin metrics often start to decrease, thereby showing signs of axonal loss and myelin degradation. Consequently, Alzheimer's disease, Parkinson's disease and multiple sclerosis are typically characterized by an increase of free water and, in later stages of these diseases, by a decrease of the apparent-fiber-density and myelin metrics.

During clinical trials, it is important to know if a particular medication is effective or not. Interestingly, when a medication is effective, the free water typically decreases (which indicates that there is less inflammation in the white matter). In some cases, an increase of the apparent-fiber-density and myelin metrics are also observed, which suggests that the medication has the effect of remyelinizing the white matter and rebuilding axons. It is hypothesized that this happens when patients are in the ‘middle stage’ of their diseases, where myelin and axons have not been irreversibly damaged.

For traumatic brain injury, immediately following brain concussion, an increase of free water is observed because of sudden neuro-inflammation and swelling. In most cases, the free water decreases after some time as the symptoms disappear. In these cases, the apparent-fiber-density and myelin metrics are stable all along. However, in some cases, the traumatic brain injury neuroinflammation becomes chronic which in turn can affect the integrity of the white matter and lead to reduction in apparent fiber density and myelin metrics.

Thus, for Alzheimer's disease, Parkinson's disease, multiple sclerosis and traumatic brain injury, there is an increase of free water followed in time by a decrease of the apparent-fiber-density and myelin metrics.

Note that where these changes occur in the brain may depend on the neurological diseases. For example, for Alzheimer's disease the changes may occur in the fornix region and in ‘Alzheimer's disease bundles.’ Alternatively, for Parkinson's disease the changes may occur in the substantia nigra and ‘Parkinson's disease bundles.’ Moreover, for multiple sclerosis the changes may occur in the vicinity of white-matter lesions and ‘multiple sclerosis bundles.’ Furthermore, for traumatic brain injury the changes may occur in a variety of locations in the brain, but there are some ‘traumatic brain injury bundles.’ Additionally, note that Alzheimer's disease, Parkinson's disease, and multiple sclerosis are often associated with an atrophy of the hippocampal volume.

While harmonization can be applied to the diffusion signal, in some embodiments it may also be applied to feature maps (such as a free-water or FW map, an apparent fiber density or AFD map, a myelin map, and/or brain regions, such as area size, circumference, volume, etc.). These feature map harmonization(s) may be deep-learning based (e.g., using a pretrained neural network) or non-deep learning base (e.g., by adjusting the image histogram to a reference histogram or using a technique such as Vanilla ComBAT, e.g., from the Dana-Farber Cancer Institute, Boston, Massachusetts).

In the Clinical ComBAT techniques, one or more operations may be performed using a pretrained neural network. For example, Clinical ComBAT may be implemented using a deep autoencoder or another type of neural network. The pretrained neural network may significantly improve the accuracy relative to existing ComBAT techniques (e.g., by 10×), thereby reducing the use of processor, memory and communication resources in or associated with a computer system (such as computer system 200 in FIG. 2).

We now further describe the Clinical ComBAT techniques. dMRI derived scalar maps have proven effective in measuring the impact of various neurodegenerative diseases and brain conditions.

FIG. 5 presents a drawing of an example of raw dMRI in accordance with an embodiment of the present disclosure. Note that CC is the Corpus Callosum, CST is the Cortical Spinal Tract, and Cg is the Cingulum.

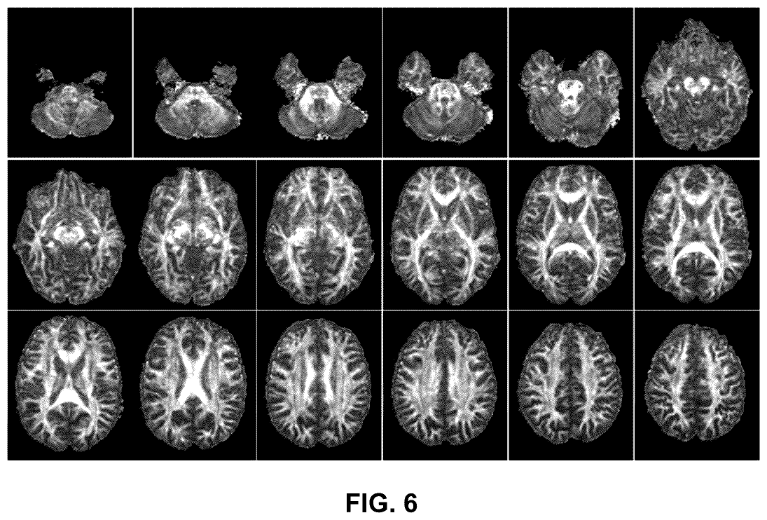

FIG. 6 presents a drawing of an example of fractional anisotropy in accordance with an embodiment of the present disclosure.

However, by their very nature, dMRIs can hinder the combination of data from multiple acquisition sites without a harmonization step to mitigate scanner-specific biases. The existing prevalent harmonization technique, ComBAT, despite its efficacy in academic and clinical studies, often faces limitations in real-life, large-scale clinical applications, particularly in personalized medicine. This is due to its reliance on assumptions that are seldom met in clinical settings, such as a linear model of data with respect to its covariates, a homogeneous population across sites, a fixed number of sites, and a large sample size to accurately estimate harmonization parameters.

In the present disclosure, we introduce Clinical ComBAT, which is designed for the nuances of real-life clinical practice. (In the present discussion, existing ComBAT techniques are sometimes referred to collectively as ‘Vanilla ComBAT.’) Our technique employs a simplified data model, uses a polynomial modeling of the data, a site-specific harmonization using a normative reference site, and a robust function to compensation for data irregularities. Additionally, it incorporates prior terms, uses a new hyperparameter tuning technique, and introduces a goodness of fit metric to evaluate harmonization quality.

ComBAT is a harmonization technique initially devised for genomics to counter batch effects. Its effectiveness in mitigating MRI acquisition effects in scalar maps from dMRI was subsequently demonstrated. Despite its widespread use, the theoretical underpinnings and implementation of ComBAT can be perplexing. To facilitate a comprehensive understanding, we detail the technique in the discussion below.

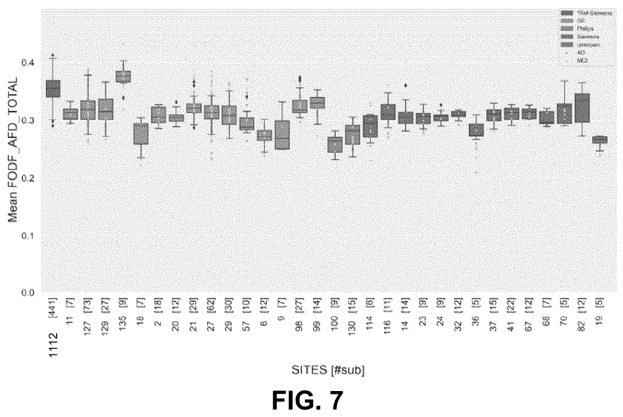

FIG. 7 presents a drawing of an example of unharmonized apparent fiber density in accordance with an embodiment of the present disclosure. The apparent fiber density in the brain region arcuate fasciculus in the left hemisphere was determined for 32 sites using the 1212 dataset. Also shown in FIG. 7 are the sites for the 1112 dataset.

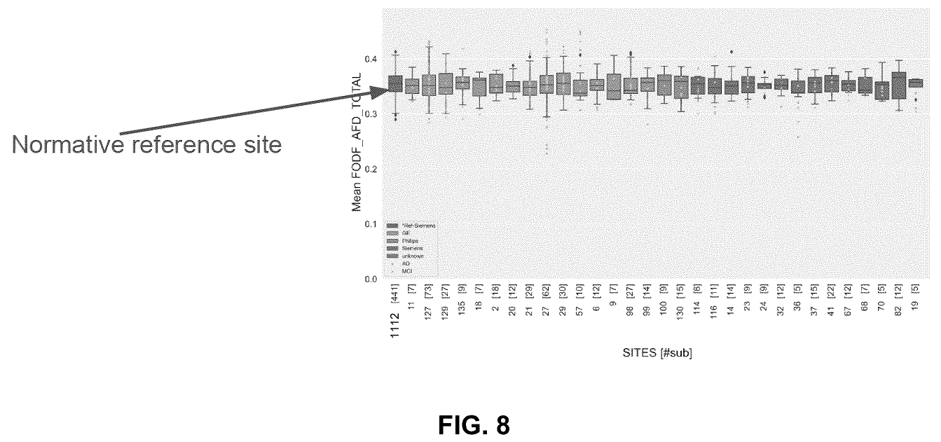

FIG. 8 presents a drawing of an example of harmonized apparent fiber density in accordance with an embodiment of the present disclosure. In FIG. 8, the 1112 dataset was used as the normative or target reference.

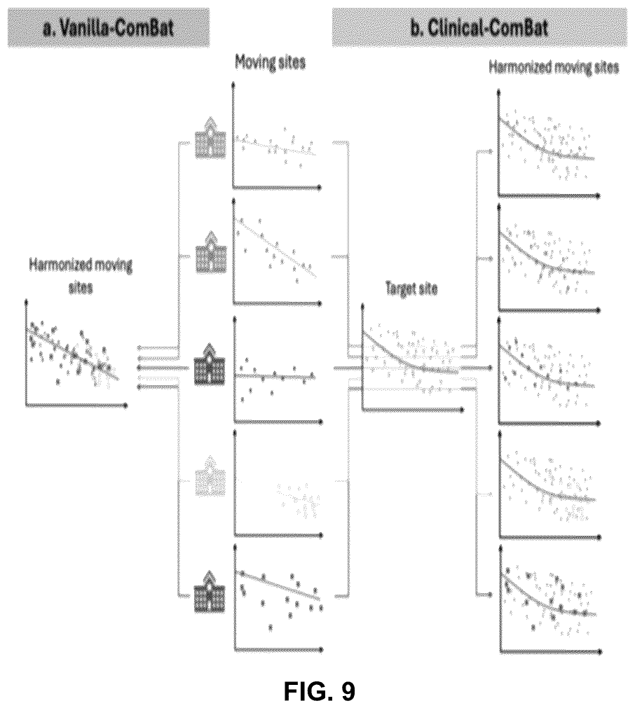

FIG. 9 presents a drawing of examples of a Vanilla (existing) ComBAT technique and a Clinical ComBAT technique in accordance with an embodiment of the present disclosure. Notably, FIG. 9 shows the operations in ComBAT. Moreover, Table 1 presents a list of ComBAT variables.

In order to address the problems with Vanilla ComBAT, we introduce Clinical ComBAT, which is designed for the practicalities of clinical use. As shown in FIG. 9, Clinical ComBAT aligns each (moving) site with a well-populated (target) normative reference, circumventing the issue of increasing site numbers. Moreover, we improved the model and replaced the linear function with a nonlinear, polynomial one. Also, using a large normative population allows for: the integration of new priors, the estimation of a degree of a polynomial function, the integration of a robust function to handle abnormal data, and the inclusion of a goodness of fit metric to assess harmonization quality.

Let Y={Y₁, Y₂, . . . , Y₁,} represent a set of I physical MRI sites, where Yᵢ={Yᵢ₁, Yᵢ₂, . . . , Y_{iJ}_{ᵢ}} are a set of Jᵢ participants whose brain has been scanned with the same MRI machine i. In voxel-wise harmonization, these participant's MR images are usually non-linearly registered to a common space, such as the MNI space. Note that the number of participants Jᵢ is site dependent, as their number varies from one site to another. Each scalar map Yᵢⱼ can be expressed as Yᵢⱼ=[yᵢⱼ₁, yᵢⱼ₂, . . . yᵢⱼᵥ], where V is the number of voxels (or regions) in the common space and y_{ijy} ∈ [symbol] .

| Context | Variable | Meaning |
|---|---|---|
| Global | i, I | Physical MRI site index. Total number of sites |
| | j, Jᵢ | Scan index for site i, Total number of scans for site i |
| | v, V | Voxel index for site i and scan j, Total number of voxols |
| | y | Set of I physical MRI sites |
| | Yᵢ | Set of Jᵢ scans of site i |
| | Yᵢⱼ | Scan j from site i |
| Unstandardized | yᵢⱼᵥ | Feature of site i, scan j, and voxel v |
| | αᵥ, \hat{α}ᵥ | Model intercept of the overall population, Estimate |
| | ${\hat{α}}_{n}^{CC}$ | Estimate of CamCan intercept |
| | xᵢⱼ, Xᵢⱼ | Yᵢⱼ vector of biological covariates, Matrix of stacked xᵢⱼ |
| | βᵥ, \hat{β}ᵥ | Regression vector for a voxel v, Estimate |
| | ${β}_{v}^{CC}$ | Estimated CamCan regression vector for a voxel v, |
| | γᵢᵥ, \hat{γ}ᵢᵥ | Additive bias of site i at the voxel v, Estimate |
| | δᵢᵥ, \hat{δ}ᵢᵥ | Multiplicative bias of site i at the voxel v, Estimate |
| | [symbol] | Random independent Gaussian noise for the feature yᵢⱼᵥ |
| | σᵥ, \hat{σ}ᵥ | Standard deviation of [symbol] , Estimate |
| | ${σ}_{v}^{CC}$ | Estimate of CamCan standard deviation |
| Standardized | z_{ifv} | Standardized value of yᵢⱼᵥ |
| | γᵢᵥ^{*} | Standardized additive bias |
| | ${γ}_{iv}^{*}$ | $Estimate of {γ}_{iv}^{*}$ |
| | ${\overline{γ}}_{iv}^{*}$ | $Estimate of {γ}_{iv}^{*} using an empirical Bayes$ |
| | ${δ}_{iv}^{*}$ | Standardized multiplicative bias |
| | ${\hat{δ}}_{iv}^{*}$ | $Estimate of {δ}_{iv}^{*}$ |
| | ${\overline{δ}}_{iv}^{*}$ | $Estimate of δ \text{?} using an empirical Bayes$ |
| | μᵢ, μ̅ᵢ | $Mean of γ \text{?} for site i , E stimate$ |
| | ${τ}_{i}^{2} , {\overline{τ}}_{i}^{2}$ | $Variance of γ \text{?} for site i , Estimate$ |
| | λᵢ, λ̅ᵢ | $Shape of δ \text{?} for site i , E stimate$ |
| | θᵢ, θ̅ᵢ | $Scale of δ \text{?} for site i , E stimate$ |
| | $\overline{G} \text{?}$ | First empirical moment of an Inverse-Gamma |
| | ${\overline{S}}_{i}^{2}$ | Second empirical moment of an Inverso-Gamma |

[symbol]

indicates data missing or illegible when filed

Table 1. List of ComBAT variables.

To mitigate site-specific biases, ComBAT employs a linear model for the image formation of each region (often voxel) ν as follows:

$${y}_{ijv} = {α}_{v} + {x}_{ij}^{T} {β}_{v} + {γ}_{iv} + {δ}_{iv} {ε}_{ijv} \qquad ( 6 )$$

where αᵥ is the model intercept of the overall population across all sites, xᵢⱼ is a vector of covariates, βᵥ is the regression coefficient vector, γᵢᵥ and δᵢᵥ are the additive and multiplicative effects of site i, and εᵢⱼᵥ is a random independent Gaussian noise with

$${ϵ}_{ijv} \sim 𝒩 ( 0 , {σ}_{v}^{2} ) .$$

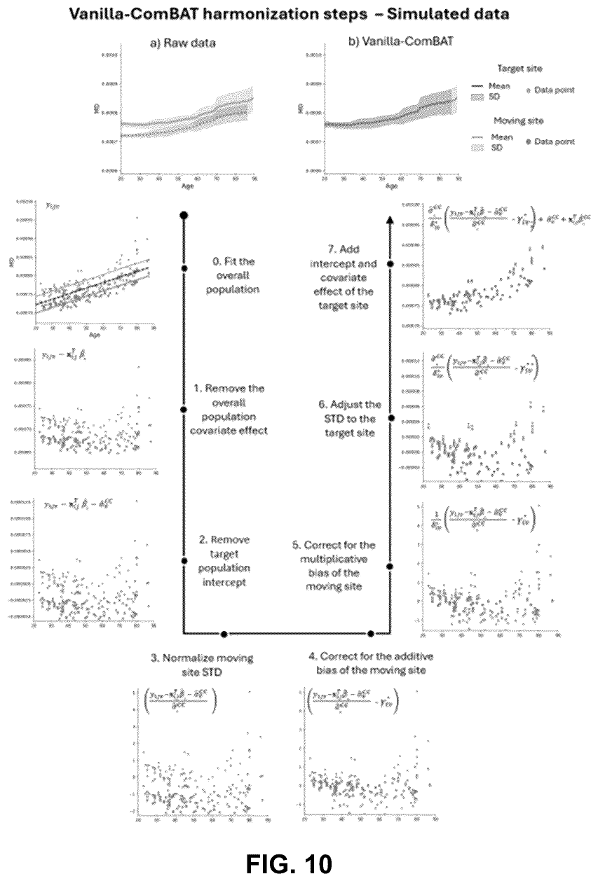

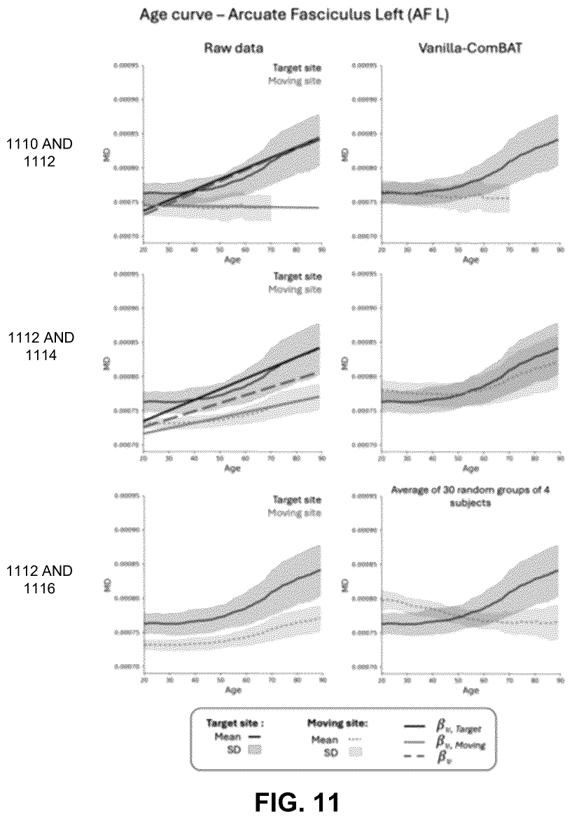

This model is depicted for two sites in FIG. 10, which presents a drawing of an example of a Vanilla ComBAT technique, and FIG. 11 presents a drawing of an example of harmonization of mean diffusivity using a Vanilla ComBAT technique. (Notably, FIG. 10 illustrates the seven operations in a typical Vanilla ComBAT harmonization of two sites underlying the effect of each variable of Eqn. 22. FIG. 10 illustrates the raw data and the harmonized data. The gray and the red curves in FIG. 10 illustrate the overall trend of the population from site 1 and site 2, whereas the scatter plots illustrate the operations leading to the harmonization of the two sites.) In FIGS. 10 and 11, the black and red lines illustrate the trend of the two populations

$$( i . e . {α}_{v} + {x}_{1 j}^{T} {β}_{v} + {γ}_{1 v} and {α}_{v} + {x}_{2 j}^{T} {β}_{v} + {γ}_{2 v} )$$

whereas the dotted line corresponds to the overall population trend i.e.

$${α}_{v} + {x}_{i j}^{T} {β}_{v} .$$

the population) is constant for all sites which, as will be demonstrated, is often incorrect and results in flawed harmonization functions. Thus, the harmonization process illustrated in FIG. 10 is an idealized version of the technique.

In FIG. 11, the MD metric is harmonized using Vanilla ComBAT between sites of healthy participants but with a different slope βᵥ. In all cases, Vanilla ComBAT leads to an erroneous harmonization. Notably, FIG. 11 shows the harmonization of the two datasets 1110 and 1112, and a different dataset 1114 and a biased version of itself. The dashed line represents the slopes and intercept computed by Vanilla ComBAT. Note that 1116 illustrates the same experiment as in 1114, but with the average of 30 groups of 4 randomly selected participants.

The primary goal of ComBAT is usually to eliminate the site-specific additive and multiplicative biases (respectively γᵢᵥ and δᵢᵥ) ensuring that the harmonized population profile conforms to the model depicted in FIG. 10,

$${y}_{ijv}^{ComBAT} = {α}_{v} + {x}_{ij}^{T} {β}_{v} + {ϵ}_{ijv} . \qquad ( 7 )$$

Because the parameters of the model αᵥ, βᵥ, γᵢᵥ and δᵢᵥ are a priori unknown, they are usually empirically estimated from the data. If these parameters can be accurately estimated (referred to as \hat{α}ᵥ, \hat{β}ᵥ, \hat{γ}ᵢᵥ and \hat{δ}ᵢᵥ), the harmonized values

$${y}_{ijv}^{ComBAT}$$

can be computed as

$${y}_{ijv}^{ComBAT} = \frac{{y}_{ijv} - {\hat{α}}_{v} - {x}_{ij}^{T} {\hat{β}}_{v} - {\hat{γ}}_{iv}}{{\hat{S}}_{iv}} + {\hat{α}}_{v} + {x}_{ij}^{T} {\hat{β}}_{v} . \qquad ( 8 )$$

One simplistic approach to estimating these parameters is through a Location and Scale (L/S) technique. Although this approach is straightforward and effective, it often has its share of limitations. These are typically addressed with the Bayesian formulation of ComBAT, which is discussed further below.

L/S ComBAT estimates the parameters \hat{α}ᵥ, \hat{β}ᵥ, \hat{γ}ᵢᵥ and \hat{δ}ᵢᵥ following a maximum likelihood of Gaussian distributions. As such, the regression vector \hat{β}ᵥ and the intercept \hat{α}ᵥ are obtained with an ordinary least-square (L/S) approach. This involves pooling data for every voxel ν from every patient j across all sites i, and use the usual L/S regression solution:

$${[ {\hat{α}}_{v} , {\hat{β}}_{v} ]}^{T} = {( {X}^{T} X )}^{- 1} {X}^{T} {Y}_{v} , \qquad ( 9 )$$

where X is the matrix of covariate vectors from every participant across all sites, and Yᵥ is a vector containing the metric associated with location ν for every participant in all sites. According to this framework, the global intercept \hat{α}ᵥ represents the average feature value at voxel ν for all images across all sites, excluding the effect of covariates, namely

$${\hat{α}}_{v} = \frac{1}{{J}_{I}} {\sum }_{ij} ( {y}_{ijv} - {x}_{ij}^{T} {\hat{β}}_{v} ) , \qquad ( 10 )$$

where J_{I} is the total number dMRI-derived metric images acquired across all sites, illustrated as the total number of gray and red points in FIG. 10.

The additive and multiplicative site-specific biases \hat{γ}ᵢᵥ and \hat{δ}ᵢᵥ are determined by taking the mean and variance of the rectified value at location ν for all images acquired at site i:

$${\tilde{γ}}_{iv} = \frac{1}{{J}_{i}} {\sum }_{j} ( {y}_{ijv} - {\hat{α}}_{v} - {x}_{ij}^{T} {\hat{β}}_{v} ) , \qquad ( 11 )$$

$${\hat{δ}}_{iv}^{2} = \frac{1}{{J}_{i} - 1} {\sum }_{j} {( {y}_{ijv} - {\hat{α}}_{v} - {x}_{ij}^{T} {\hat{β}}_{v} - {\tilde{γ}}_{iv} )}^{2} , \qquad ( 12 )$$

When the total number of images J_{I} acquired across all sites is sufficient, the maximum likelihood estimate of \hat{β}ᵥ and \hat{α}ᵥ from Eqns. 9 and 10 can be considered unbiased. However, for any site i with a low number of acquired images (a common scenario in clinical practice) \hat{γ}ᵢᵥ and \hat{δ}ᵢᵥ are biased and thus should not be relied upon.

One common solution to that problem is to estimate \hat{γ}ᵢᵥ and \hat{δ}ᵢᵥ following a maximum a posteriori formulation while keeping as is Eqns. 9 and 10 for estimating \hat{β}ᵥ and \hat{α}ᵥ. This leads to the most-used version of ComBAT, which is called Vanilla ComBAT in the remainder of the present disclosure.

Vanilla ComBAT typically begins by standardizing the data, which involves subtracting the population intercept, regression weight and variance, as depicted in FIG. 10 and represented mathematically as

$${z}_{ijv} = \frac{{y}_{ijv} - {\hat{α}}_{v} - {x}_{ij}^{T} {\hat{β}}_{v}}{{\hat{σ}}_{v}} , \qquad ( 13 )$$

where the variance is determined as

$${\hat{σ}}_{v}^{2} = \frac{1}{{J}_{I}} {\sum }_{i j} {( {y}_{i j v} - {\hat{α}}_{v} - {x}_{i j}^{T} {\hat{β}}_{v} - {\hat{γ}}_{i v} )}^{2} , \qquad ( 14 )$$

and \hat{γ}ᵢᵥ is calculated following Eqn. 11.

At this stage, the bias of each site (the vertical distance between the origin and the red and blue dashed line in FIG. 10) is denoted as

$${γ}_{i v}^{*}$$

and the variance of each site is

$${δ}_{i v}^{2 *} .$$

These two variables are usually estimated by maximizing their posterior distributions rather than their likelihood distributions. According to Bayes' theorem, the posteriors π(.) of

$${γ}_{i v}^{*} and {δ}_{i v}^{2 *}$$

can be expressed as

$$P ( {γ}_{i v}^{*} \vert {z}_{lv} , {δ}_{i v}^{2 *} ) ∝ P ( {z}_{iv} \vert {γ}_{iv}^{*} , {δ}_{i v}^{2 *} ) P ( {γ}_{i v}^{*} ) , \qquad ( 15 )$$

$$P ( {δ}_{i v}^{2 *} \vert {z}_{i v} , {γ}_{i v}^{*} ) ∝ P ( {z}_{i v} \vert {γ}_{i v}^{*} , {δ}_{i v}^{2 *} ) P ( {δ}_{i v}^{2 *} ) , where \qquad ( 16 )$$

$$P ( {z}_{iv} \vert {γ}_{iv}^{*} , {δ}_{iv}^{2 *} ) = 𝒩 ( {γ}_{iv}^{*} , {δ}_{iv}^{2 *} )$$

is the likelihood-Gaussian,

$$P ( {γ}_{i v}^{*} ) = 𝒩 ( {μ}_{i} , {τ}_{i}^{2} )$$

is the prior-Gaussian, and

$$P ( {δ}_{i v}^{2 *} ) = IG ( {λ}_{i} , {θ}_{i} )$$

is the prior-Inverse Gamma.

The hyperparameters of the prior distributions μᵢ, τᵢ², λᵢ, and θᵢ are usually estimated based on the moment of these distributions. Specifically, μᵢ and τᵢ² may be derived from the first and second moment of a Gaussian distribution, namely

$$\begin{matrix}{\overline{μ}}_{i} = \frac{1}{V} {\sum }_{v} {\hat{γ}}_{iv}^{*} \\ {\overline{τ}}_{i}^{2} = \frac{1}{V - 1} {\sum }_{v} {( {\hat{γ}}_{iv}^{*} - {\overline{u}}_{i} )}^{2}\end{matrix} \qquad ( 17 )$$

where V is the number of brain regions (or voxels) and

$${\hat{γ}}_{iv}^{*} = \frac{1}{{J}_{i}} {\sum }_{j} {z}_{ijv}$$

is the region-wise and site-wise model intercept of the standardized data and should not be confused with \hat{γ}ᵢᵥ from Eqn. 11.

To estimate λᵢ and θᵢ, one usually must first calculate the voxel-wise and site-wise standardized variance

$${\hat{δ}}_{iv}^{2 *} = \frac{1}{{J}_{i} - 1} {\sum }_{j} {( {z}_{ijv} - )}^{2} .$$

This variance shall not be confused wise standardized variance with

$${\hat{δ}}_{iv}^{2}$$

from Eqn 12. The empirical mean and variance of

$${\hat{δ}}_{iv}^{2 *}$$

across all voxels is computed as

$${\overline{G}}_{ι} = \frac{1}{V} {\sum }_{v} {\hat{δ}}_{iv}^{2 *} and \overline{{S}_{ι}^{2}} = \frac{1}{V - 1} {\sum }_{v} {( {\hat{δ}}_{iv}^{2 *} - {\overline{G}}_{i} )}^{2} .$$

Then, by equating G̅ᵢ and

$${\overline{V}}_{i}^{2}$$

to the first and second theoretical moments of the inverse gamma distribution, we get,

$${\overline{S}}_{i}^{2} = \frac{{\overline{G}}_{ι} = \frac{{θ}_{i}}{{λ}_{{i}_{{θ}_{i}^{2}}} - 1}}{{( {λ}_{i} - 1 )}^{2} ( {λ}_{i} - 2 )} \qquad ( 18 )$$

which can be rearranged to estimate the two hyperparameters,

$${\overline{λ}}_{\text{?}} = \frac{{\overline{G}}_{\text{?}}^{2} + 2 {\overline{S}}_{\text{?}}^{2}}{{\overline{S}}_{\text{?}}^{2}} , {\overline{θ}}_{\text{?}} = \frac{{\overline{G}}_{\text{?}}^{3} + {\overline{GS}}_{\text{?}}^{2}}{{\overline{S}}_{\text{?}}^{2}} \qquad ( 19 )$$

$$\text{?} \text{indicates text missing or illegible when filed}$$

Now that the hyperparameters of the likelihood and prior distributions have been estimated, by expanding Eqns. 15 and 16 and integrating them with Eqn. 17 and (19), we arrive at the mathematical expectation of the posterior distributions, which yields the estimates of

$${γ}_{iv}^{*} and {δ}_{i v}^{2 *}$$

$${\overline{γ}}_{\text{?}}^{*} = \dot{𝔼} ( {γ}_{\text{?}}^{*} \vert {z}_{\text{?}} , {δ}_{\text{?}}^{2 *} ) = \frac{{J}_{\text{?}} {\overline{τ}}_{\text{?}}^{2} {\hat{γ}}_{\text{?}}^{*} + {\overline{δ}}_{\text{?}}^{2 *} {\overline{μ}}_{\text{?}}}{{J}_{\text{?}} {\overline{τ}}_{\text{?}}^{2} + {\overline{δ}}_{\text{?}}^{2 *}} \qquad ( 20 )$$

$${\overline{δ}}_{\text{?}}^{2 *} = \dot{𝔼} ( {δ}_{\text{?}}^{2 *} \vert {z}_{\text{?}} , {γ}_{\text{?}}^{*} ) = \frac{{\overline{θ}}_{\text{?}} + \frac{1}{2} {\sum }_{j} {( {z}_{ijv} - {\overline{γ}}_{iv}^{*} )}^{2}}{\frac{{J}_{\text{?}}}{2} + {\overline{λ}}_{i} - 1} \qquad ( 21 )$$

$$\text{?} \text{indicates text missing or illegible when filed}$$

Given the interdependency of Eqns. 20 and 21,

$${\overline{γ}}_{i v}^{*} and {\overline{δ}}_{i v}^{2 *}$$

are often computed through an iterative process. This may start with a reasonable initial value for

$${\overline{δ}}_{i v}^{2 *} ( e . g . , {\hat{δ}}_{i v}^{2 *} ) ,$$

followed by the calculation of

$${γ}_{iv}^{*} ,$$

and then re-estimation of

$${\overline{δ}}_{i v}^{2 *}$$

using the new

$${γ}_{i v}^{*}$$

value, and so forth. This cycle can be repeated until convergence is achieved.

After all parameters of Eqn. 7 have been empirically estimated, data harmonization is performed as follows:

$${y}_{\text{?}}^{ComBat} = \frac{{{σ}^{\text{?}}}_{v}}{{\overline{δ}}_{\text{?}}^{*}} ( {z}_{\text{?}} - {\overline{γ}}_{\text{?}}^{*} ) + {{α}^{\text{?}}}_{\text{?}} + {x}_{\text{?}}^{T} {{β}^{\text{?}}}_{\text{?}} \qquad ( 22 )$$

$$\text{?} \text{indicates text missing or illegible when filed}$$

Despite its popularity, Vanilla ComBAT often has its limitations. As such, over the years, several derived versions of ComBAT have been proposed to improve the original approach. Most of these improved versions tackle the core assumptions of ComBAT. For example, ComBAT-GAM counters the linear assumption of ComBAT using spline-based generalized additive models (GAM). M-ComBAT harmonizes data to a specific reference (or target) site rather than a global average. B-ComBAT employes Monte Carlo bootstrapping to robustly estimate the parameters in ComBAT, whereas Longitudinal ComBAT integrates intra-subject temporal information. CoVBat additionally corrects for covariance effects besides variance and mean.

Vanilla ComBAT and its subsequent versions are primarily designed for use in the context of clinical and/or academic research studies. Here, ‘studies’ (or ‘trials’) are defined as research initiatives where participants (human or otherwise) undergo specific interventions under a predetermined research plan with data collected across multiple sites following stringent protocols. ‘Studies’ may also encompass retrospective research on public datasets. However, the assumption-laden ComBAT harmonization for study data is typically not well-suited to the dynamism of real-life clinical practice. This necessitates a specialized harmonization approach for clinical settings, which we introduce as Clinical ComBAT.

Clinical ComBAT is designed for the nuances of real-life clinical practice. It employs a simplified data model, uses a polynomial modeling of the data, a site-specific harmonization using a normative reference site, and/or a robust function to compensation for data irregularities. Additionally, it incorporates novel prior terms, and/or introduces a goodness of fit metric to evaluate harmonization quality.

We now discuss harmonization in clinical practice. While not widely documented, the harmonization requirements for data collected in research studies versus day-to-day clinical practice are often distinct. We have identified nine core differences between these two settings, as outlined in Table 2. The most significant distinction lies in their primary objectives. Research studies focus on analyzing population trends, characterizing diseases, identifying biomarkers, or developing diagnostic and prognostic techniques. Note that studies typically involve a homogeneous population, often compared to a reference group. In contrast, clinical practice is centered on personalized medicine, where the data of each individual are processed and analyzed independently of other participants scanned at the same site. Therefore, the harmonization requirement in clinical practice is usually oriented towards comparing the metrics of an individual to those of a normative reference, akin to comparing a child's height and body mass index against pediatric growth charts.

TABLE 2
Differences between research studies and clinical practice.

| | Clinical studies | Clinical practice |
|---|---|---|
| Application | Population study | Personalized medicine |
| Acquisition period | Limited | Unlimited |
| Nb MRI scanner | Fixed | Increases over time |
| Nb participants per site | Fixed | Increases over time (very |
| | | low at the beginning) |
| MRI drifting | Little or none | Inevitable |
| Harmonization need | When all data has been acquired | As soon as possible |
| Medical condition | Similar within and across sites | Different within and across |
| | (known for each participant) | sites (often unknown for |
| | | each patient) |
| Age bracket | Similar span across sites | Variable |
| Acquisition protocol | Similar across sites | Different across sites |

The second difference relates to the acquisition period. Research studies are inherently finite, concluding with a fixed number of sites and participants. In contrast, real-life clinical practice usually involves a continuous influx of data over an indefinite period for each site. Furthermore, from a software service provider's standpoint, the number of data-contributing sites may expand at an unpredictable rate over time. The open-ended acquisition timeline in clinical settings also means that the MRI signal is prone to drift, a factor often not problematic in research studies. Such drifting can result from hardware aging or updates to software and/or hardware.

An additional distinction is the timing of data harmonization. In clinical research, harmonization is typically a one-time process, executed after all data has been collected and curated. Conversely, in day-to-day clinical practice, harmonization typically occurs each time new data is acquired.

The final difference lies in the heterogeneity of the data. Research studies enforce strict acquisition protocols and select participants based on various covariates like physical condition, lifestyle, age, sex, etc., resulting in a consistent data profile across sites. In contrast, day-to-day clinical practice deals with a highly heterogeneous patient population, without a specific selection process. Moreover, the acquisition protocols can vary significantly from one site to another, adding to the complexity of data harmonization in clinical environments.

Given these factors, day-to-day clinical practice necessitates a harmonization technique tailored to its specific realities. First, scanner drift-due to the normal aging of scanners—and periodic software and hardware updates usually necessitate regular updates to the harmonization function of MRI clinical scanners. However, clinics and hospitals often hesitate to regularly acquire new data solely for updating harmonization, because of various financial, ethical, and staff availability constraints. As such, a proper harmonization should normally adapt to drifting without requiring the acquisition of specific data. Second, the harmonization function usually must also perform well with limited data, which is common when a site begins scanning its initial patients. It should also be robust against data spanning a limited age range and corrupted data from patients with undocumented diseases. Furthermore, when new sites are integrated into the network, the harmonization process should not require recalibrating the entire network, as would be the case with indiscriminate application of Vanilla ComBAT.

Given the realities of clinical practice, several drawbacks emerge from the indiscriminate use of Vanilla ComBAT. The first issue is its linear assumption regarding data distribution (c.f. Eqns. 6 and 7). Although data may exhibit linear distribution in certain scenarios, this is not universally applicable, as illustrated by the Mean Diffusivity age distribution in FIG. 11. As will be discussed further below, a more effective harmonization technique is one that can automatically adjust to the trend of the data, which is often nonlinear.

Another significant issue with Vanilla ComBAT is its assumption that the slope (parameter βᵥ) of the distribution remains consistent across all sites. While this might not be a problem when the acquisition protocol of all sites has been carefully tuned, this assumption is often wrong between sites exhibiting very different signals, as demonstrated in FIG. 11. For example, the slopes of two populations in two datasets 1110 and 1112 (represented by black and blue dashed lines) differ notably. Given the non-linear nature of the MD metric with respect to age, and because these populations cover different age ranges (20 to 60 years for 1110 and 19 to 90 years for 1114), the slopes for both sites diverge substantially. These slope discrepancies are further amplified by significant additive and multiplicative biases between the sites. Additionally, the larger population size of 1112 (441 participants) compared to 1110 (132 participants) means the slope derived from Eqn. 9 leans more towards 1112 than 1110, leading to the inappropriate dash line for 1110 in FIG. 11, which underpins the flawed harmonization shown in FIG. 11.

FIG. 11 exemplifies this issue with the harmonization of a modified version of 1114 against itself. Assuming for this data the linear model in Eqn. 7, simulating the effects of multiplicative and additive biases (γᵢ and δᵢ) yields the linear model \hat{γ}ᵢⱼᵥ=yᵢⱼᵥδᵢ+γᵢ, or more explicitly

$${\hat{y}}_{i j v} = {α}_{v} {δ}_{i} + {γ}_{i} + {x}_{i j}^{T} {β}_{v} {δ}_{i} + {ϵ}_{ijv} {δ}_{i} . \qquad ( 23 )$$

This indicates that any multiplicative bias affects the population slope (here βᵥδᵢ) contradicting the Vanilla ComBAT assumption of uniform slope across sites. In FIG. 11, with applied multiplicative and additive biases of 0.5 and 0.0008 to 1114, it is evident that Vanilla ComBAT is inadequate for re-harmonizing the data.

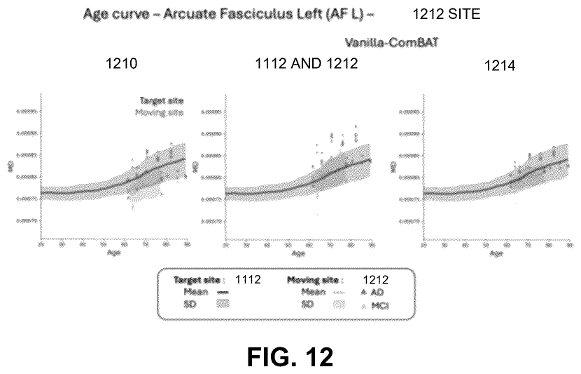

FIG. 12 presents a drawing of an example of harmonization of mean diffusivity of two different sites using a Vanilla ComBAT technique in accordance with an embodiment of the present disclosure. Notably, FIG. 12 illustrates raw data 1210, Vanilla ComBAT harmonization of the healthy subject in two datasets 1112 and 1212, and Vanilla ComBAT harmonization of all participants (healthy plus Alzheimer's Disease or AD and Moderate Cognitive Impairment or MCI) with the health participants in the other dataset 1214.

A further significant limitation is that the intercept and slope parameters (αᵥ and βᵥ) are estimated using the maximum likelihood approach of Eqn. 9 without any regularization prior. This leads to considerable variability when only a small number of participants are used to calculate the ComBAT parameters. This issue is depicted for 1116 in FIG. 11, where the biased version of the dataset 1116 is harmonized onto the original dataset 1112. As opposed to dataset 1114 where the harmonization parameters were derived using all 441 participants, only 4 participants were used in 1116. This procedure was repeated 30 times, each with a different set of participants. The average resulting curves, shown in FIG. 11 for 1116, exhibit significant difference.

Another limitation stems from Vanilla ComBAT's assumption that the data to be harmonized across all sites come from a homogeneous patient population. In practice, the populations scanned across multiple imaging centers are often heterogeneous, as they do not undergo a uniform selection process. This discrepancy becomes apparent when harmonizing clinical sites onto a normative reference target, which ideally should comprise only healthy controls (HC). An example of this is shown in FIG. 12, where the data from the 1212 dataset is harmonized with the 1112 dataset, which acts as the normative target. While the 1112 dataset includes only healthy control (HC) participants, the 1212 dataset contains a mix of healthy control, Mild Cognitive Impairment, and Alzheimer's disease patients.

Raw data 1210 in FIG. 12 highlights the differences between the two populations. The blue and gray-shaded areas indicate the healthy control distribution for both sites, whereas the turquoise and orange triangles represent respectively the Alzheimer's disease and Mild Cognitive Impairment participants from the first site. In 1212, the harmonization outcome is presented when only healthy control from the first site were used to derive the ComBAT parameters in comparison to second-site data. In contrast, 1214 illustrates the harmonization process for both healthy control and Alzheimer's disease participants from the first site. Harmonizing using only healthy control participants correctly segregates the Alzheimer's disease and Mild Cognitive Impairment participants, placing the Alzheimer's disease participants away from the normal population curve, which does not happen when both healthy control and Alzheimer's disease and Mild Cognitive Impairment participants are included in the harmonization. This discrepancy underscores the flaw in assuming patient population homogeneity during the harmonization process. Of course, it is possible to include these pathologies in the covariate vector \vec{x}ᵢⱼ along with age and sex, allowing for their consideration in the estimation of βᵥ. However, there is currently no standardized reference encompassing the distribution of multiple pathologies encountered in routine clinical settings. Moreover, in clinical practice, patient pathologies are frequently undisclosed, given that the primary aim of scans is to aid in diagnosing suspected neurological conditions.

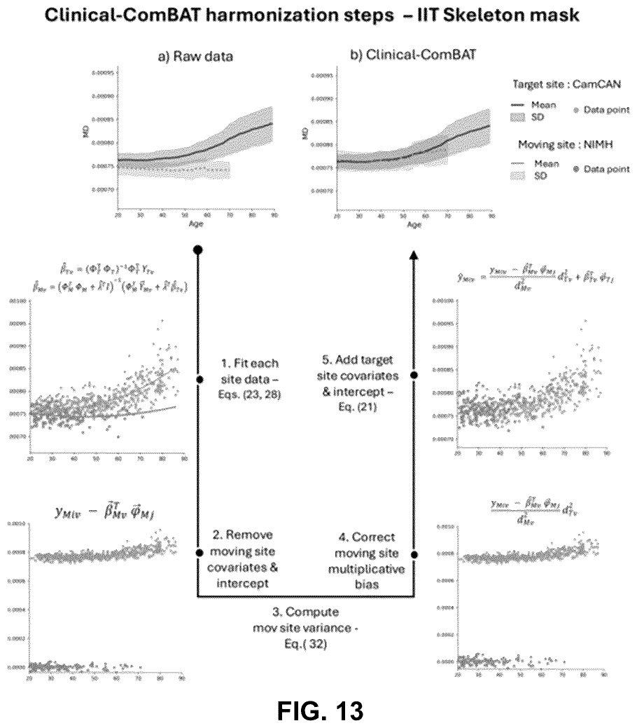

Clinical ComBAT introduces eleven modifications compared to Vanilla ComBAT, each tailored to better suit the nuances of clinical practice. The primary focus of Clinical ComBAT is to facilitate the comparison of patient data against a normative model, which is an important aspect of clinical applications. FIG. 13 presents a drawing of an example of harmonization using a Clinical ComBAT technique in accordance with an embodiment of the present disclosure. Notably, FIG. 13 depicts the operations in the harmonization procedure of Clinical ComBAT. The first notable feature of Clinical ComBAT is its strategy of harmonizing each site with a specific clinical target site. A similar approach of pairwise harmonization has been previously explored with M-ComBAT. However, Clinical ComBAT enhances this concept by ensuring that the target site contains a sufficiently large and diverse healthy control (HC) population to establish a robust normative model. It is predicated on the diversity of the subject distribution of the target site, encompassing a wide range of ages, handedness, and a balanced representation of genders.

The pairwise harmonization approach in Clinical ComBAT effectively addresses two challenges. First, it avoids the complexity of needing to harmonize an ever-growing number of sites simultaneously, as each site is harmonized independently. Secondly, it resolves the problem with Vanilla ComBAT, which could inadvertently modify the harmonization parameters for already calibrated sites when applied indiscriminately to a new site.

FIG. 13 presents a drawing of an example of harmonization of a moving site and a target site using a Clinical ComBAT technique in accordance with an embodiment of the present disclosure. Notably, 1310 is the raw data, and 1312 is the harmonized data. The grey (target site) and the red (moving site) lines illustrate the overall trend of the population from site 1 and site 2. (1-4) are the operations leading to the harmonization of the two sites.

Additionally, pairwise harmonization simplifies the data formation equation used in Vanilla ComBAT (see Eq. (6)). At the core of most ComBAT techniques is the assumption that each dataset includes two additive biases (a population bias αᵥ and a site bias γᵢᵥ) and two multiplicative biases (a population bias σᵥ and a site bias δᵢᵥ). These four parameters are intended to represent the variations of each site from an overall unbiased reference population. Because Clinical ComBAT independently harmonizes each moving site, the data formation model can be streamlined to two biases. First, the model being a linear equation, Clinical ComBAT combines ay and γᵢᵥ into a single site-wise additive bias, denoted as bᵢᵥ. Additionally, because the multiplication of a normally distributed variable

$${ϵ}_{ijv} ∼ 𝒩 ( 0 , {σ}_{v}^{2} )$$

by δᵢᵥ still results in a normal distribution with variance (σᵥδᵢᵥ)², our model adopts a single multiplicative bias, denoted as dᵢᵥ. This modification leads to the following simplified site-wise data formation equation:

$${y}_{i j v} = {b}_{i v} + {x}_{i j} {β}_{v} + {d}_{iv} {ϵ}_{ijv} , \qquad ( 24 )$$

where i represents the site index i∈{Moving, Target} and ϵᵢⱼᵥ˜ [symbol] (0,1). This new data formation equation stands as the second improvement with respect to Vanilla ComBAT.

A third distinction arises with the slope parameter βᵥ in Vanilla ComBAT, which is the same across every site. Clinical ComBAT introduces a subtle but crucial modification by adding a site index i, resulting in βᵢᵥ. Consequently, each site has its own distinct slope, an adjustment that is critical for compensating in specific scenarios, as will be demonstrated in the results section.

The fourth distinction in Clinical ComBAT is the nonlinearity introduced in the Vanilla ComBAT model. This nonlinearity is achieved using a polynomial basis function of positive degree P, expressed as

$$φ ( {\vec{x}}_{i j} ) = {( {\vec{x}}_{i j}^{T} \cdot {\vec{x}}_{i j} + 1 )}^{P} \qquad ( 25 )$$

denoted as \vec{φ}ᵢⱼ for notation simplicity. Notably, regardless of the degree P, the expansion of this polynomial always includes a constant term, which allows the additive bias bᵢᵥ to be effectively integrated into it. Consequently, the data formation equation is revised to

$${y}_{ijv} = {\vec{φ}}_{i j}^{T} {\vec{β}}_{i v} + {d}_{iv} {ϵ}_{ijv} . \qquad ( 26 )$$

Here, each site is characterized by a specific set of weights \vec{β}ᵢᵥ, along with a single multiplicative bias dᵢᵥ and an additive bias that is incorporated into \vec{β}ᵢᵥ.

With the pairwise harmonization framework of Clinical ComBAT and according to Eqn. 26, the harmonization of a moving site M onto a target normative reference T requires the estimation of four terms: two weight vectors \vec{β}_{Mv} and \vec{β}_{Tv} and two multiplicative biases d_{Mv} and d_{Tv}.

For the target site, like Vanilla ComBAT, \vec{β}_{Tv} is computed by maximizing a likelihood Gaussian distribution of the target data. Doing so boils down to minimizing the following L2 loss function

$${\vec{β}}_{T v} = arg {max}_{\vec{β}} \frac{1}{{J}_{T}} {\sum }_{i = 1}^{{J}_{T}} {( {y}_{Tjv} - {\vec{φ}}_{Tj}^{T} {\vec{β}}_{T v} )}^{2} , \qquad ( 27 )$$

which may be solved with the following close form solution

$${\vec{β}}_{Tv} = {( {Φ}_{T}^{T} {Φ}_{T} )}^{- 1} {Φ}_{T}^{T} {Y}_{Tv} , \qquad ( 28 )$$

where Φ_{T} is a matrix containing the concatenated covariable vectors \vec{φ}_{Tj} of all target participants, and Y_{Tv} is a vector containing the values y_{Tjv} of the target site. While this formulation is similar to that of Eqn. 9, it only contains data from the target site whereas Eqn. 9 contains data from every site.

A fifth difference with Vanilla ComBAT is on the computation of

$${d}_{Tv}^{2} .$$

With the hypothesis that the target site is well populated, the estimation of

$${d}_{Tv}^{2}$$

does not require an a priori term as in Eqns. 15 and 16. Instead,

$${d}_{Tv}^{2}$$

can be estimated following a maximum likelihood which is a much simpler alternative to the iterative solution in Vanilla ComBAT derived from the use of an inverse gamma prior term (c.f. Eqns. 20 and 21). Instead, d_{Tv}² is obtained following a maximum likelihood of a Gaussian distribution which leads to the calculation of the simple variance of the rectified data,

$${d}_{Tv}^{2} = \frac{1}{{J}_{T}} {\sum }_{j = 1}^{{J}_{T}} {( {y}_{Tjv} - {\vec{φ}}_{Tj}^{T} {\vec{β}}_{Tv} )}^{2} . \qquad ( 29 )$$

Because the moving site may contain a limited number of data, we proceed with the estimation of \vec{β}_{Mv} and d_{Mv} with a maximum a posteriori (MAP) formulation. Unlike Vanilla ComBAT (the sixth difference) which models \vec{β}_{Mv} with a likelihood distribution, we model it with a posterior distribution involving the product of a Gaussian likelihood and prior:

$$P ( {\vec{β}}_{Mv} \vert {D}_{Mv} ) ∝ P ( {D}_{Mv} \vert {\vec{β}}_{Mv} ) P ( {\vec{β}}_{Mv} ) \qquad ( 30 )$$

where D_{Mv}={Y_{Mv}, X_{M}} contains the list of all values and covariables of the moving site at location ν and where

$$P ( {D}_{Mv} \vert {\vec{β}}_{Mv} ) = 𝒩 ( {Φ}_{M}^{T} {\vec{β}}_{iv} , {\sum }_{l} ) ( likelihood - Gaussian ) \qquad ( 31 )$$

$$P ( {\vec{β}}_{Mv} ) = 𝒩 ( {\vec{β}}_{Tv} , {\sum }_{0} ) ) ( Prior - Gaussian ) . \qquad ( 32 )$$

The goal of the prior is to make sure \vec{β}_{Mv} does not differ too much from \vec{β}_{Tv}, the regression weights of the target site. This is often important when a site accounts for a very low number of participants. Note that maximizing the posterior P(\vec{β}_{Mv}|D_{Mv}) leads to the following close-form solution:

$${\vec{β}}_{Mv} = {( {Φ}_{M}^{T} {Φ}_{M} + {\vec{λ}}^{T} I )}^{- 1} ( {Φ}_{M}^{T} {\vec{Y}}_{Mv} + {\vec{λ}}^{T} {\vec{β}}_{Tv} ) , \qquad ( 33 )$$

where I is the identity matrix and \vec{λ}∈ [symbol] ^{p+} is a regularization vector. As can be seen, for \vec{λ}=\vec{0}, the regression of \vec{β}_{Mv} is a maximum likelihood estimation as in Eqn. 28. Moreover, as the regularization increases, more weight is put on \vec{β}_{Tv}, and, when \vec{λ} becomes large enough, \vec{β}_{Mv}≈\vec{β}_{Tv}.

A seventh difference with Vanilla ComBAT comes from our maximum a posteriori formulation for estimating the moving site variance

$${d}_{Mv}^{2} .$$

For Clinical ComBAT, the posterior is given by

$$P ( {d}_{Mv}^{2} \vert {z}_{Mv} ) ∝ P ( {z}_{Mv} \vert {d}_{Mv}^{2} ) P ( {d}_{Mv}^{2} ) , \qquad ( 34 )$$

where z_{Mv} is the set of all rectified values of the moving site, namely

$${z}_{Mjv} = {y}_{Mjv} - {\vec{φ}}_{Mj}^{T} {\vec{β}}_{Mv} ∀ j .$$

Like Vanilla ComBAT, the likelihood distribution follows a Gaussian distribution. However, the prior is a Gamma (instead of an inverse Gamma) distribution:

$$P ( {z}_{Mv} \vert {d}_{Mv}^{2} ) = 𝒩 ( 0 , {d}_{Mv}^{2} ) ( likelihood - Gaussian ) , \qquad ( 35 )$$

$$P ( {λ}_{Mv} ) = G ( {λ}_{Mv} \vert {a}_{0} , {b}_{0} ) ( prior - Gamma ) , \qquad ( 36 )$$

where

$${λ}_{Mv} = \frac{1}{{d}_{Mv}^{2}}$$

is the inverse of the variance also called precision. By incorporating the variance of the target site

$${d}_{Tv}^{2}$$

in the Gamma prior, maximizing the posterior leads to the following solution:

$${d}_{Mv}^{2} = \frac{{J}_{M} {\hat{d}}_{Mv}^{2}}{{J}_{M} + {v}_{0}} + \frac{{v}_{0} {d}_{Tv}^{2}}{{J}_{M} + {v}_{0}} \qquad ( 37 )$$

where J_{M} is the number of data in the moving site,

$${\hat{d}}_{Mv}^{2}$$

is the empirical variance of the rectified moving site

$$\frac{1}{{J}_{M}} {\sum }_{j = 1}^{{J}_{M}} {( {y}_{Mjv} - {\vec{φ}}_{Mj}^{T} {\vec{β}}_{Mv} )}^{2}$$

and ν₀∈ [symbol] ⁺ is an hyperparameter. This equation comes with an intuitive flavor. Indeed, when the moving site lacks data, indicated by J_{M}=0, the variance of the moving site

$${d}_{Mv}^{2}$$

defaults to match the variance of the target site

$${d}_{Tv}^{2} .$$

As the quantity of data of the moving site increases,

$${d}_{Mv}^{2}$$

transitions to a weighted average between

$${\hat{d}}_{M v}^{2} and {d}_{Tv}^{2} .$$

At some point, when

$${J}_{M} = {v}_{0} , {d}_{Mv}^{2}$$

becomes the exact average between

$${\hat{d}}_{Mv}^{2} and {d}_{Tv}^{2} .$$

Beyond this point, when

$${J}_{M} ≫ {v}_{0} , {d}_{Mv}^{2}$$

closely approximates to

$${\hat{d}}_{Mv}^{2} .$$

In addition to its simplicity, Eqn. 37 does not require an iterative process and has the target variance as a prior instead of data taken across the brain.

We now describe the harmonization process. At the heart of Clinical ComBAT are two techniques. The first, named Clinical ComBAT-fit, processes data and covariates for a specified region v collected at the target and moving sites. The data from the target site, D_{Tv}, includes {(y_{T1v}, \vec{x}_{T1}), (y_{T2v}, \vec{x}_{T2}), . . . , (y_{TN}_{_{T}}ᵥ, \vec{x}_{TJ}_{_{T}})} and similarly, the moving site data, D_{Mv}, includes {(y_{M1v}, \vec{x}_{M1}), (y_{M2v}, \vec{x}_{M2}), . . . , (y_{MN}_{_{M}}ᵥ, \vec{x}_{MJ}_{_{M}})}. From these, it calculates the four harmonization parameters

$$\{ {d}_{Mv}^{2} , {\vec{β}}_{Mv} , {d}_{Tv}^{2} , {\vec{β}}_{Tv} \} .$$

The second technique is called Clinical ComBAT-Apply. It uses a set of J_{M} data from the moving site and harmonizes with the target site. Detailed explanations and mathematical frameworks of these techniques are outlined in Tables 3 and 4.

TABLE 3
Clinical ComBAT-Fit

| | Input: D_{Tv} ={(y_{T1v}, \vec{x}_{T1}), (y_{T2v}, \vec{x}_{T2}), ... , (y_{TN}_{_{T}}ᵥ, \vec{x}_{TJ}_{_{T}}) | // Target site data |
|---|---|---|
| | Input: D_{Mv} = {(y_{M1v}, \vec{x}_{M1}), (y_{M2v}, \vec{x}_{M2}), ... , (y_{MN}_{_{M}}ᵥ, \vec{x}_{MJ}_{_{M}})} | // Moving site data |
| | Input: P, \vec{λ}, V₀ | // Hyperparameters |
| | $Output : \{ {d}_{M v}^{2} , {\vec{β}}_{Mv} , {d}_{T v}^{2} , {\vec{β}}_{Tv} \}$ | // harmonization parameters |
| | // Target site parameters | |
| 1 | ${\vec{φ}}_{T j} ← {( {\vec{x}}_{Tj}^{T} \cdot {\vec{x}}_{T j} + 1 )}^{P} ∀ j$ | Eqn. 24 |
| 2 | Φ_{T} ← stack every vector \vec{φ}_{Tj} in a matrix | |
| 3 | ${\vec{β}}_{Tv} = {( {Φ}_{T}^{T} {Φ}_{T} )}^{- 1} {Φ}_{T}^{T} {Y}_{Tv}$ | Eqn. 28 |
| 4 | ${d}_{T v}^{2} = \frac{1}{{J}_{T}} {\sum }_{j = 1}^{{J}_{T}} {( {y}_{Tjv} - {\vec{φ}}_{Tj}^{T} {\vec{β}}_{Tv} )}^{2}$ | Eqn. 29 |
| | // Moving site parameters | |
| 5 | ${\vec{φ}}_{Mj} ← {( {\vec{x}}_{Mj}^{T} \cdot {\vec{x}}_{Mj} + 1 )}^{P} ∀ j$ | Eqn. (24 |
| 6 | Φ_{M} ← stack every vector \vec{φ}_{Mj} in a matrix | |
| 7 | ${\vec{β}}_{Mv} = {( {Φ}_{M}^{T} {Φ}_{M} + {\vec{λ}}^{T} I )}^{- 1} ( {Φ}_{M}^{T} {\vec{Y}}_{Mv} + {\vec{λ}}^{T} {\vec{β}}_{Tv} )$ | Eqn. 33 |
| 8 | ${\hat{d}}_{M v}^{2} = \frac{1}{{J}_{M}} {\sum }_{j = 1}^{{J}_{M}} {( {y}_{Mjv} - {\vec{φ}}_{Mj}^{T} {\vec{β}}_{Mv} )}^{2}$ | |
| 9 | ${d}_{M v}^{2} = \frac{{J}_{M} {\hat{d}}_{M v}^{2}}{{J}_{M} + {v}_{0}} + \frac{{v}_{0} {d}_{Tv}^{2}}{{J}_{M} + {v}_{0}}$ | Eqn. 37 |
| 10 | $Return \{ {d}_{M v}^{2} , {\vec{β}}_{Mv} , {d}_{T v}^{2} , {\vec{β}}_{Tv} \}$ | |

TABLE 4
Clinical ComBAT-Apply

| | Input: D_{Mv} = {(y_{M1v}, \vec{x}_{M1}), (y_{M2v}, \vec{x}_{M2}), ... , (y_{MN}_{_{M}}ᵥ, \vec{x}_{MJ}_{_{M′}})} | // Moving site data |
|---|---|---|
| | Input: P | // Hyperparameter |
| | Output: {ŷ_{M1v}, ŷ_{M2v}, ... ŷ_{MN}_{_{M}}ᵥ} | // harmonized |
| | values | |
| 1 | for j = 1 ... J_{M}, do | |
| 2 | $\vert {\vec{φ}}_{Mj} ← {( {\vec{x}}_{Mj}^{T} \cdot {\vec{x}}_{Mj} + 1 )}^{P}$ | Eqn. 24 |
| 3 | $\vert {\hat{y}}_{Miv} = \frac{{y}_{Miv} - {\vec{β}}_{Mv}^{T} {\vec{φ}}_{Mj}}{{d}_{Mv}^{2}} {d}_{Tv}^{2} + {\vec{β}}_{Tv}^{T} {\vec{φ}}_{Tj}$ | Eqn. 26 |
| 4 | Return {ŷ_{M1v}, ŷ_{M2v}, ... ŷ_{MJ}_{_{M}}ᵥ} | |

Additional improvements in Clinical ComBAT include goodness of fit quality control. FIGS. 11 and 12 demonstrate that Vanilla ComBAT (and its variants) are susceptible to harmonization errors. These errors often result in a harmonized population distribution that is poorly aligned to the target population. This issue is clearly depicted in FIG. 11, where the red-shaded harmonized distribution leans downward compared to the gray-shaded target population. Such discrepancies in harmonization can lead to misinterpretation of the data by healthcare providers.

From this perspective, an eighth different with Vanilla ComBAT is a quality control system designed to identify abnormal shifts in a set of harmonized data relative to a target population. Several heuristic techniques can be implemented, such as counting the instances where harmonized data points fall two (or more) standard deviations away from the reference population. A flag would be raised when these deviations exceed a predefined threshold.

In the case of Clinical ComBAT, a population overlap score is computed to gauge the extent of overlap between the target and harmonized populations. Initially, from the original raw datasets D_{Tv} and D_{Mv} and using the Clinical ComBAT-Fit technique, we derive the harmonization parameters

$$\{ {d}_{Mv}^{2} , {\vec{β}}_{Mv} , {d}_{Tv}^{2} , {\vec{β}}_{Tv} \} .$$

Subsequently, applying Clinical ComBAT-Apply to D_{Mv} (or on any other dataset from the moving site) produces a set of harmonized data called D̅_{Mv}.

From this point on, the objective is to determine how much the harmonized population D̅_{Mv} overlaps the target population Dry. This may be achieved by rectifying both populations using the target parameter vector \vec{β}_{Tv}:

$$\begin{matrix}{z}_{Tjv} = {y}_{Tjv} - {\vec{φ}}_{Tj}^{T} {\vec{β}}_{Tv} & ∀ ( {y}_{Tjv} , {\vec{φ}}_{Tj}^{T} ) ∈ {D}_{Tv}\end{matrix} \qquad ( 38 )$$

$$\begin{matrix}{\hat{z}}_{Mjv} = {\hat{y}}_{Mjv} - {\vec{φ}}_{Mj}^{T} {\vec{β}}_{Tv} & ∀ ( {\hat{y}}_{Mjv} , {\vec{φ}}_{Mj}^{T} ) ∈ {D}_{Mv} .\end{matrix} \qquad ( 39 )$$

By removing the effect of the covariates, this rectification process allows us to assume that zᵢⱼᵥ is independent from \vec{x} and that P(z_{Tjv}|\vec{x}_{Tj})=P(z_{Tjv}) and P(\hat{z}_{Mjv}|\vec{x}_{Mj})=P(\hat{z}_{Mjv}).

The distance between univariate distribution functions can be computed using various statistical metrics and divergence measures, including: total variation distance, Hellinger distance, Lévy-Prokhorov metric, Wasserstein metric, Mahalanobis distance, Amari distance, integral probability metrics, KL divergence, Jensen-Shannon divergence, Bhattacharyya distance, Rényi divergence, and/or f-divergence. In Table 7, we illustrate the use of the Bhattacharyya distance which, in the case of two Gaussian distributions such as in ours, results into the close form solution of line 5 in Table 7.

TABLE 5
Harmonization-Qc

| Input \hat{D}_{Mv} = {(ŷ_{M1v}, \vec{x}_{M1}), (ŷ_{M2v}, \vec{x}_{M2}), ... , (ŷ_{MN}_{_{M}}ᵥ, \vec{x}_{MJ}_{_{M′}})} | // Harmonized |
|---|---|
| data | |
| D_{Tv} = {(y_{T1v}, \vec{x}_{T1}), (y_{T2v}, \vec{x}_{T2}), ... , (y_{TN}_{_{T}}ᵥ, \vec{x}_{TJ}_{_{T′}})} | // Target site data |
| thr (default = 0.04) | // Threshold |
| \vec{β}_{Tv} | // Target |

| | |
|---|---|
| | site parameter |
| | Output Qc flag |
| | // data rectification |
| 1 | ${Z}_{T} = \{ {z}_{Tjv} = {y}_{Tjv} - {\vec{φ}}_{Tj}^{T} {\vec{β}}_{Tv} \vert ∀ ( {y}_{Tjv} , {\vec{φ}}_{Tj}^{T} ) ∈ {D}_{Tv} \}$ |
| 2 | ${Z}_{M} = \{ {\hat{z}}_{Mjv} = {\hat{y}}_{Mjv} - {\vec{φ}}_{Mj}^{T} {\vec{β}}_{Tv} \vert ∀ ( {\hat{y}}_{Mjv} , {\vec{φ}}_{Mj}^{T} ) ∈ {D}_{Mv} \}$ |
| | // Compute the Bhattacharyya distance |
| 3 | ${μ}_{T} = mean ( {Z}_{T} ) , {σ}_{T}^{2} = var ( {Z}_{T} )$ |
| 4 | ${μ}_{M} = mean ( {Z}_{M} ) , {σ}_{M}^{2} = var ( {Z}_{M} )$ |
| 5 | ${d}_{B} = \frac{1}{4} \frac{{( {μ}_{T} - {μ}_{M} )}^{2}}{{σ}_{T}^{2} + {σ}_{M}^{2}} + \frac{1}{2} ln ( \frac{{σ}_{T}^{2} + {σ}_{M}^{2}}{2 {σ}_{T} {σ}_{M}} )$ |
| 6 | if d_{B} > thr then // a default value for thr is 0.04 |
| 7 | return False |
| 8 | else |
| 9 | return True |

The nineth difference with Vanilla ComBAT is the rejection of outliers. Data acquired in real-life conditions is rarely obtained from healthy individuals. Instead, it is typically collected from subjects with conditions that may affect the measured dMRI signal. Furthermore, the condition of the subject is often unknown at the time of scanning, as the primary purpose of an MRI scan is to aid in diagnosis. Consequently, the moving data D_{Mv} can often be corrupted by outlier measures. If used as-is, this can lead to situations such as the one illustrated in FIG. 12 where the harmonization squashes together the healthy and non-healthy measures along the normative target model.

One solution is to reduce as much as possible the influence of outlier data. One way of doing so is by removing data that are spreading far away from the center of gravity of the distribution. This can be done by clipping data that are more than a few standard deviations away from the mean or the median of the population or whose p-value is below a certain threshold. As an alternative, one can also use techniques based on the median absolute deviation or other estimators like the s, and the _{Q}_{ₙ} estimators.

However, because outlier data are typically due to persons suffering from a medical condition, the outlier typically fall on one side of the distribution (e.g. in FIG. 12 the Alzheimer's disease subjects are all above the average). As such, a blind clipping may end up removing as many normal data than outliers. As an alternative, one might iteratively remove extreme data on the side the distribution is skewed towards, up until when the remaining data exhibit a better Gaussian shape, such as when it passes a normality test.

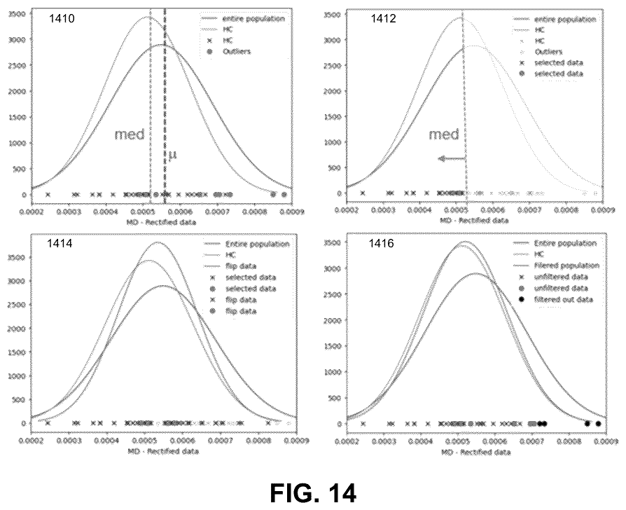

FIG. 14 presents a drawing of an example of outlier rejection in accordance with an embodiment of the present disclosure. Notably, FIG. 14 illustrates an outlier rejection technique with data lying mostly on one site of the distribution. 1410 shows a population made of healthy controls (in blue) and outliers (in red) with the entire population distribution being shifted toward the outliers. Moreover, 1412 shows data lying on the left of the median are considered healthy controls. Furthermore, 1414 shows that flipping the selected data results into a distribution that is closer to the healthy control distribution. Additionally, 1416 shows that removing the right most extreme data leads to a distribution close to that of the healthy controls.

One way of doing so is illustrated in FIG. 14 for the mean diffusivity metric. Starting with rectified moving data

$${Z}_{M} = \{ {\hat{z}}_{Mjv} = {\hat{y}}_{Mjv} - {\vec{φ}}_{Mj}^{T} {\vec{β}}_{Mv} \vert ∀ ( {\hat{y}}_{Mjv} , {\vec{φ}}_{Mj}^{T} ) ∈ {D}_{Mv} \} ,$$

1410 illustrates the healthy controls and outlier data as blue and red points. As can be seen, the distribution of the entire population (healthy controls and outliers) is skewed towards the right, i.e., on the side of the red outlier data. One way of removing the outliers is to compute the median of the entire population and consider the data on the left of the median to be mostly composed of healthy controls (1412). Then, by flipping these data around the median and fitting on it a Gaussian distribution (the green aka Flip distribution in 1414) we get a distribution that approximate that of the healthy controls. Then, one can iteratively remove the right-most data (illustrated by the black dots) up until when the mean of the entire population falls on the left of the flip distribution as shown by 1416.

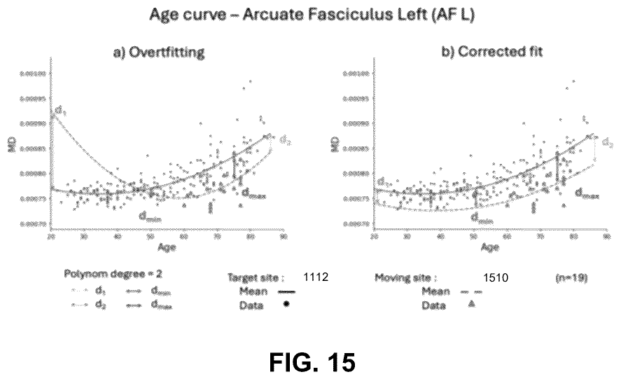

FIG. 15 presents a drawing of examples of different polynomial models of degree 3 in accordance with an embodiment of the present disclosure. The gray dots with their associated gray curve model are from the 1112 dataset and the orange ones are from sample of 19 participants from a 1510 dataset. Overfitting of the moving model obtained with \vec{λ}-0 diverges for the 20-40 age range. Moreover, the result of an auto-tuning technique illustrates that the shape of the orange model does not deviate too much from that of the gray model.

We now discuss hyperparameter auto-tuning. The tenth difference with Vanilla ComBAT is the ability for the disclosed Clinical ComBAT technique to automatically tune its most influential hyperparameter. For Clinical ComBAT, that hyperparameter is \vec{λ} in Eqn. 33. The size of this hyperparameter vector dictates the extent to which the model weights, denoted by \vec{β}_{Mv}, will diverge from the target site weights \vec{β}_{Tv}. At one extreme, when \vec{λ}→\vec{0}, \vec{β}_{Mv} aligns closely with the moving data, potentially leading to overfitting. This is more likely when the moving dataset is small, contains outliers, or is limited to a narrow age range, or when the polynomial degree P of Eqn. 24 is too high. FIG. 15 provides an example, displaying target data (gray dots) against raw moving data (orange triangles). In this example, the amount of data in the moving site is low and spans over a limited age range. The gray curve represents the target model fit:

$${\vec{β}}_{T v}^{T} {\vec{φ}}_{j} ∀ j ∈ [ 18 , 90 ]$$

calculated with Eqn. 28, while the orange dotted curve shows the moving model fit:

$${\vec{β}}_{Mv}^{T} {\vec{φ}}_{j} ∀ j ∈ [ 18 , 90 ]$$

derived from Eqn. 33. Both curves are polynomials of degree P equal to 3. On the left, for \vec{λ}=\vec{0}, the orange dotted curve visibly overfits, deviating significantly from the gray curve. Conversely, with a carefully adjusted \vec{λ}, the orange dotted curve on the right strikes a balance between fitting the data and resembling the gray target curve.

Assuming that the vector \vec{β}_{Tv} has the correct form, our objective is to identify the appropriate \vec{λ} vector that ensures the orange curve aligns closely with the gray curve while accurately representing the orange moving data. This task is quantified using four distinct distance measures between the two curves, as illustrated in FIG. 15. The first two measures, dₘᵢₙ and dₘₐₓ represent the minimum and maximum distances between the curves across all voxels ν for the age range present in the moving data:

$${d}_{m i n} = min ( {\vec{β}}_{Tv}^{T} {\vec{φ}}_{j} - {\vec{β}}_{Mv}^{T} {\vec{φ}}_{j} ) ∀ j ∈ {x}_{M} , \qquad ( 40 )$$

$${d}_{m ax} = max ( {\vec{β}}_{Tv}^{T} {\vec{φ}}_{j} - {\vec{β}}_{Mv}^{T} {\vec{φ}}_{j} ) ∀ j ∈ {x}_{M} . \qquad ( 41 )$$

The other two distances d₁ and d₂, are the minimum and maximum inter-curve distances across all voxels ν for the full age range.

The optimization of A can be expressed as

$$\vec{λ} = arg {min}_{\vec{λ}} sign ( {d}_{m i n} {τ}_{{1}^{-}} {d}_{1} ) + sign ( {d}_{2} - {d}_{m ax} {τ}_{2} ) + 2 , \qquad ( 42 )$$

where τ₁ ∈[0,1] and τ₂≥1 are predefined values, and the sign function is defined as

$$sign ( α ) = \{ \begin{matrix}1 & if α \geq 0 \\ - 1 & otherwise .\end{matrix} \qquad ( 43 )$$

Note that when τ₁=τ₂=1, the optimized \vec{λ} vector results in an orange dotted curve perfectly parallel to the gray target curve. Furthermore, since τ₁ is positive, the two curves cannot intersect as in FIG. 13.

TABLE 6
Hyperparameter auto-tuning

| | |
|---|---|
| Input: D_{Tv} = {(y_{T1v}, \vec{x}_{T1}), (y_{T2v}, \vec{x}_{T2}), ... , (y_{TN}_{_{T}}ᵥ, \vec{x}_{TJ}_{_{T′}})} | // Target site data |
| Input: D_{Mv} = {(y_{M1v}, \vec{x}_{M1}), (y_{M2v}, \vec{x}_{M2}), ... , (y_{MN}_{_{M}}ᵥ, \vec{x}_{MJ}_{_{M′}})} | // Moving site data |
| Input: P, v₀, τ₁, τ₂, k, λₘᵢₙ | // Hyperparameters |
| Output: \vec{λ} | // harmonization |

| | |
|---|---|
| | parameters |
| | // Initialization of \vec{λ} |
| 1 | $\{ {d}_{Mv}^{2} , {\vec{β}}_{Mv} , {d}_{Tv}^{2} , {\vec{β}}_{Tv} \} = ClinicalComBAT - Fit ( P , \vec{0} , {v}_{0} , {D}_{Tv} , {D}_{Mv} )$ |
| 2 | ${\vec{λ}}_{0} = \vert \frac{{\vec{β}}_{Tv}^{[ 0 ]}}{{\vec{β}}_{Tv}} \vert$ |
| | // 1D Search technique |
| 3 | found = False |
| 4 | λ = λₘᵢₙ |
| 5 | while found = = False do |
| 6 | \vec{λ} = λ, \vec{λ}₀ |
| 7 | $\{ {d}_{Mv}^{2} , {\vec{β}}_{Mv} , {d}_{Tv}^{2} , {\vec{β}}_{Tv} \} = ClinicalComBAT - Fit ( P , \vec{λ} , {v}_{0} , {D}_{Tv} , {D}_{Mv} )$ |
| 8 | Compute dₘᵢₙ, dₘₐₓ, d₁, d₂ |
| 9 | if sign(dₘᵢₙ τ₁ − d₁) + sign(d₂ − dₘₐₓ τ₂) + 2 = = 0 then |
| | Eqn.(35) |
| 10 | found = True |
| 11 | else: |
| 12 | λ = λ × k // k is a multiplicative step site > 1 |
| 13 | return \vec{λ} |

Finding a suitable \vec{λ} vector that satisfies the optimization condition of Eqn. 42 can be achieved using a technique such as the simplex optimizer. Alternatively, one may initialize \tilde{λ} with a normalized version \vec{β}_{Tv} and then incrementally adjust a multiplicative term until the optimization criterion is met. This approach simplifies the optimization process to one dimension, with details provided in Table 6.

Please note that the hyper-parameter ν₀ may also be estimated with the same auto-tuning approach. However, our empirical experiments reveal that a value of ν₀=5 produces good results in every case we tested.

Note that computing harmonization parameters with a very small number of participants (e.g. 10 or less) of moving data can result in a transfer function that is poorly suited for accurately harmonizing future data. This issue can be particularly pronounced when the initial data used to compute these parameters has a limited range of covariates. For example, when the data is from participants who are all of similar age (e.g., between 20 and 25), the harmonization parameters may not be effective for future participants with a significantly different age range (e.g., between 70 and 90).

The solution to this problem is the eleveth difference with Vanilla ComBAT and its variants. As such, the disclosed Clinical ComBAT technique pools the harmonization parameters

$$\{ {d}_{M v}^{2} , {\vec{β}}_{Mv} , {d}_{Tv}^{2} , {\vec{β}}_{T v} \}$$

or the data from a community of sites that have been properly harmonized beforehand. When the number of these community sites is sufficiently large, it can be assumed that the population distribution of the moving site is similar to at least one of these third-party sites. Based at least in part on this hypothesis, the strategy may be to identify the closest third-party sites whose distribution of covariates matches that of the moving site and use their harmonization parameters and/or data to harmonize the moving site. This approach forms the basis of the Community-ComBAT harmonization technique, as described in Table 7.

In this technique, the distance between the moving site D_{Mv} and a third-party site D_{cv} is measured by the Bhattacharyya distance of the rectified data. Other statistical distances, such as the Kullback-Leibler divergence, total variation distance, Hellinger distance, Jensen-Shannon divergence, or any Earth mover distances, could also be used. Moreover, to prevent the more heavily populated sites from having an overwhelming influence on the ComBAT fit (e.g., in line 7 of Table 6), one can resample the distribution of every site in order to have an equal amount of data among the k closest sites in the community.

TABLE 7
Community-ComBAT

| | Input: D_{Tv} = {(y_{T1v}, \vec{x}_{T1}), (y_{T2v}, \vec{x}_{T2}), ... , (y_{TN}_{_{T}}ᵥ, \vec{x}_{TJ}_{_{T′}})} | // Target site data |
|---|---|---|
| | Input: D_{cv} = {D_{cv1}, D_{cv2}, D_{cv3}, ... , D_{cvM}} | // M Community site data |
| | Input: D_{Mv} = {(y_{M1v}, \vec{x}_{M1}), (y_{M2v}, \vec{x}_{M2}), ... , (y_{MN}_{_{M}}ᵥ, \vec{x}_{MJ}_{_{M′}})} | // Moving site data |
| | Input: P, \vec{λ}, v₀, k, nb_sub | // Hyperparameters |
| | $Output : \{ {d}_{Mv}^{2} , {\vec{β}}_{Mv ,} {d}_{Tv}^{2} , {\vec{β}}_{Tv} \}$ | // harmonization parameters |
| | // Initialization of \vec{λ} | |
| 1 | Site_dist = { } | |
| 2 | For i = 1 to M do | |
| 3 | // Compute the Bhattacharyya distance | |
| 4 | d_{B} = Harmonization-Qc(D_{Mv}, D_{cvi}, \vec{β}_{cvi}) | // section 2.7.4 |

| | |
|---|---|
| 5 | Site_dist ← Site dist ∪ (d_{B}, D_{cvi},) |
| | // Harmonize the data of the closest sites onto the target site |
| 6 | Dₖᵥ ← pool a fix number of nb_sub data of the k closest sites according to Site_dist |
| 7 | $\{ {d}_{Mv}^{2} , {\vec{β}}_{Mv} , {d}_{Tv}^{2} , {\vec{β}}_{Tv} \} = Clinical ComBAT - Fit ( {D}_{Tv} , {D}_{kv} , P , \vec{λ} , {v}_{0} )$ |
| 8 | $return \{ {d}_{Mv}^{2} , {\vec{β}}_{Mv} , {d}_{Tv}^{2} , {\vec{β}}_{Tv} \}$ |

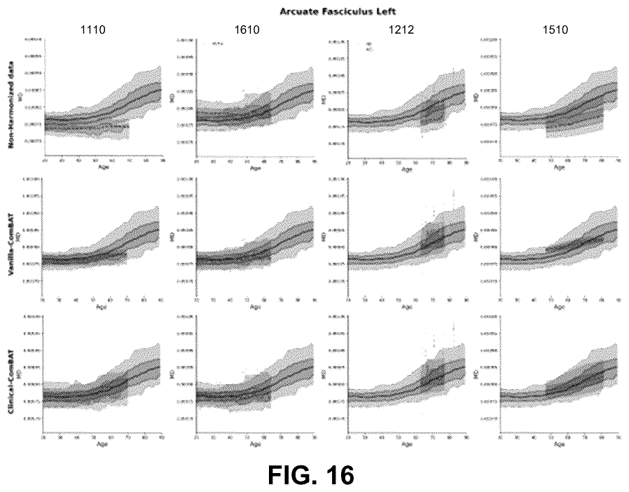

FIG. 16 presents a drawing of examples of harmonization of mean diffusivity of the arcuate fasciculus in the left hemisphere in the 1110, 1212, 1510 and 1610 datasets using a Vanilla ComBAT technique and a Clinical ComBAT technique in accordance with an embodiment of the present disclosure. The lines indicate the 95th, 75th, 50th, 25th, and 5th percentiles.

For off-the-shelf Clinical ComBAT, our experiments suggest that the polynomial degree for modeling should be determined by the target site data. The minimal degree that provides an adequate goodness of fit should be chosen (e.g., identified using the Bhattacharyya distance). Specifically, mean diffusivity, fractional anisotropy, and apparent fiber density may be suitably fitted with a polynomial degree of 2.

The variance estimation parameter ν₀ controls the variance estimation of the moving site data

$${d}_{M v}^{2} .$$

A too low ν₀ value can lead to misestimation and incorrect variance correction when data points are sparse. Conversely, a too high ν₀ value fixes the moving site variance to the target site variance, failing to correct the multiplicative bias of the moving site. Our experiments on the mean diffusion metrics white matter bundles suggest ν₀=5 provides a reliable estimation of

$${d}_{M v}^{2} .$$

When sufficient data points are available,

$${d}_{M v}^{2}$$

is progressively influenced by the target site data.

The hyperparameters τ₁ and τ₂ control the predictive capability of the model for unseen age data. Our experiments suggest that τ₁=0.5 and τ₂=2 strike a good balance between the moving site data-driven fit and providing reasonable model predictions for unseen data. Values closer to one tend to fix the polynomial curve shape to the target site curve, while excessively high values may yield unrealistic model predictions.

We conducted a series of experiments to underline the robustness of Clinical ComBAT compared to Vanilla ComBAT. Results were obtained on the data from the 1110, 1212, 1510 and 1610 datasets.

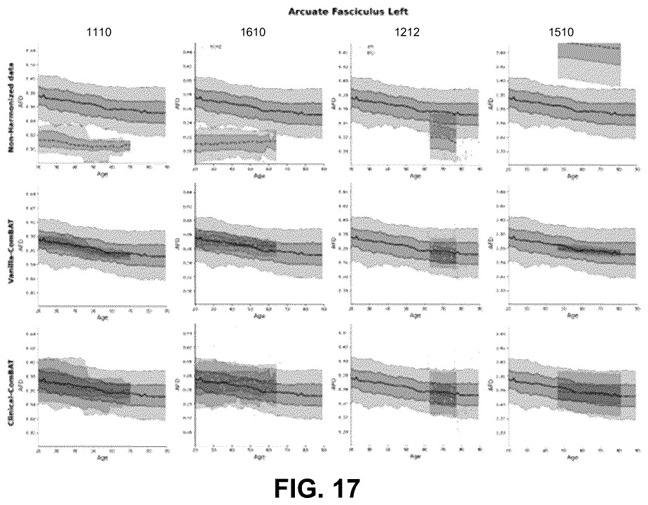

The harmonization results for mean diffusivity (FIG. 16) and apparent fiber density (as shown in FIG. 17, which presents a drawing of examples of harmonization of apparent fiber density using a Vanilla ComBAT technique and a Clinical ComBAT technique in accordance with an embodiment of the present disclosure) of the arcuate fasciculus in the left hemisphere, using Vanilla ComBAT and Clinical ComBAT, are presented for the 1110, 1212, 1510 and 1610 datasets (with, respectively, 119 health controls, 73 healthy controls, 11,382 healthy controls and 80 healthy controls). In FIG. 17, the lines indicate the 95th, 75th, 50th, 25th and 5th percentiles. Moreover, the first row of FIGS. 16 and 17 illustrate the pre-harmonized data. These figures underscore the necessity of data harmonization, as the non-harmonized data distributions are misaligned with the target site. While Vanilla ComBAT improves alignment to the target site, it often overestimates the multiplicative bias, artificially concentrating the data towards the mean of the target distribution and reducing the distinction between pathological subjects and healthy controls (e.g., the second and third sites). Conversely, Clinical ComBAT provides a more accurate estimation of the multiplicative bias, resulting in a similar distribution for healthy controls and better delineation of pathological subjects.

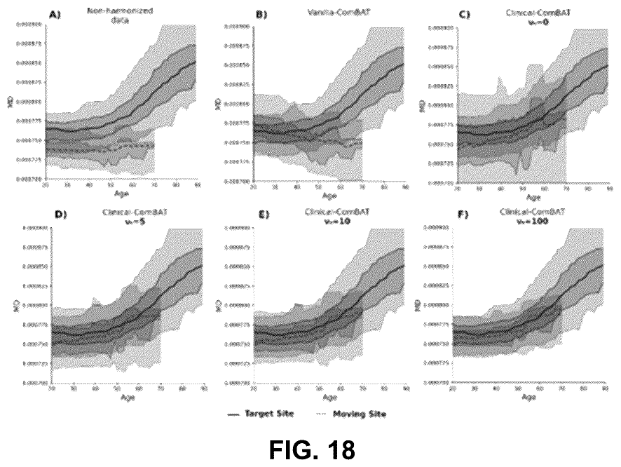

FIG. 18 presents a drawing of an example of harmonization using a Clinical ComBAT technique in accordance with an embodiment of the present disclosure. Notably, FIG. 18 illustrates the effect of the variance a priori of Clinical ComBAT on a limited number of healthy controls. Here, we used data from ten randomly selected healthy control subjects from the 1110 dataset and tested different values of ν₀ (see Eqn. 37). Note that the parameter ν₀ of Clinical ComBAT controls the weight of the a priori on the variance estimation of the moving site

$$( {d}_{M v}^{2} ) .$$

In FIG. 18, panel A shows the non-harmonized data. The harmonization results using Vanilla ComBAT are shown in Panel B, using ten randomly selected healthy controls. Panels C, D, E, F show the harmonization results on the same ten participants using increasing ν₀ values (see Eqn. 37). With ν₀=0, the standard deviation of the moving site is overestimated and overcorrected. Panels D, E show the standard deviation of the moving site aligned with the standard deviation of the target site (ν₀=5, ν₀=10). The last column demonstrates an underestimation of the standard deviation because of an excessively strong a priori from the target site (ν₀=100).

In the panel C in FIG. 18, with ν₀=0,

$${d}_{M v}^{2}$$

is underestimated, leading to an under correction of the multiplicative bias, as indicated by the larger percentile lines compared to the target site. Panels D and E in FIG. 18 shows

$${d}_{M v}^{2}$$

aligned with

$${d}_{T v}^{2} ( {v}_{0} = 5 , {v}_{0} = 10 )$$

whereas panel f in FIG. 18 demonstrates an overestimation of

$${d}_{M v}^{2}$$

due to an excessively strong a priori from the target site (ν₀=100). A high value of ν₀ with few moving site subjects effectively fixes the standard deviation of the moving site

$${d}_{M v}^{2}$$

to that of the target site

$${d}_{T v}^{2} ,$$

preventing correction the multiplicative bias of the moving site data.

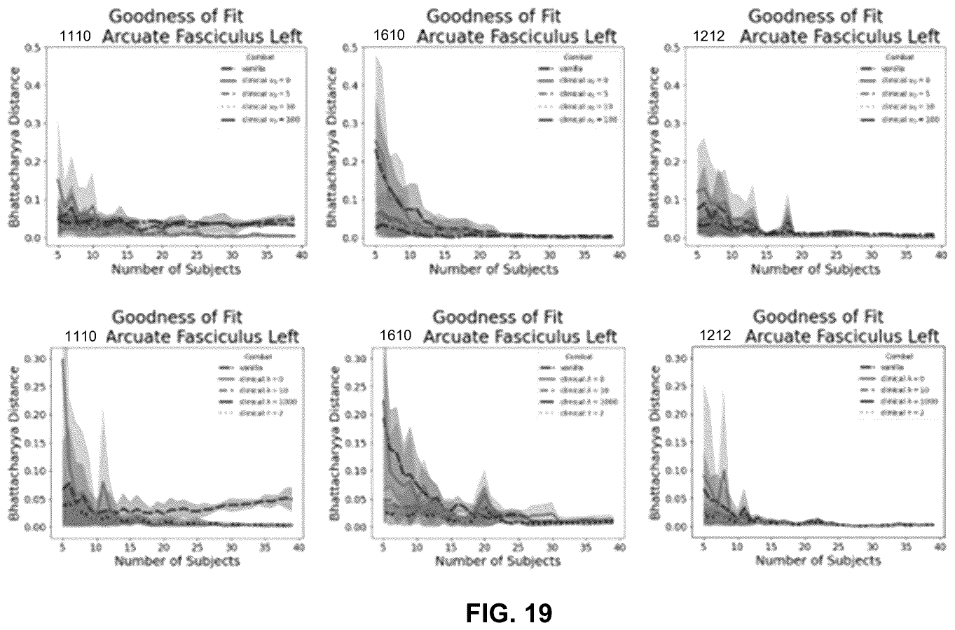

FIG. 19 presents a drawing of an example of a goodness of fit (see Table 5) of the harmonization of mean diffusivity of the arcuate fasciculus in the left hemisphere using a Clinical ComBAT technique in accordance with an embodiment of the present disclosure. The plots are shown for various numbers of healthy controls for the 1110, 1212 and 1610 datasets, and different hyper-parameter values λ and ν₀. The Clinical ComBAT model is fitted ten times with random healthy control subjects for each number of subjects. The lines show the mean Bhattacharyya distance (the smaller the better), and the shaded areas indicate one standard deviation. At the top of FIG. 19, and with a fix regularization term λ=10, the solid line (teal, ν₀=0) show poor performance with low number of subjects. The dashed (green, ν₀=5) and dotted (orange, ν₀=10) lines show similar excellent results regardless of the number of subjects. The dot-dashed line (purple, ν₀=100) show improved results with low number of subjects compared to harmonization with ν₀=0. However, as the number of subjects increases, the strong a priori leads to an increased distance when more subject are available. The bottom panel shows results for four different values of λ and the auto-tuning of λ (curve clinical τ₂=2) for a fix ν₀=5. The best results are obtained with the auto-tuning technique which outperforms Vanilla ComBAT. Vanilla ComBAT (the red or orange dashed lines) with hyperparameter auto-tuning with τ₁=0.5 and τ₂=2 tends to show weaker harmonization results than Clinical ComBAT, especially for ν₀=5 or ν₀=10. Note that the Clinical ComBAT model is fitted ten times for each number of subjects.

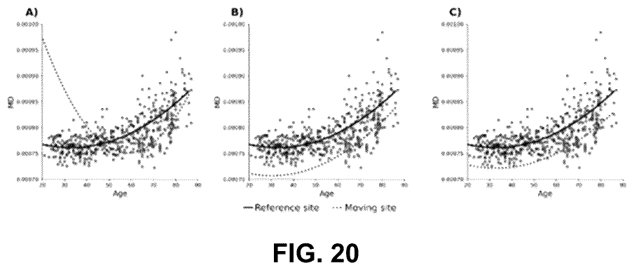

FIG. 20 presents a drawing of an example of a hyperparameter tuning when dealing with a moving site whose healthy control subjects span a limited age range (from age 64 to 80) in accordance with an embodiment of the present disclosure. In FIG. 20, the solid black line represents the second-order model fitted to the target healthy control data (black dots), while the dashed blue line represents the second-order model fitted to the healthy controls of the 1212 dataset (blue dots) using Clinical ComBAT. Panel A shows the model fitted without regularization. Panels B and C show the model fitted with automatic hyperparameter tuning using τ₁=0.25, τ₂=4, and τ₁=1, τ₂=1, respectively.

Although panel A in FIG. 20 shows a good model fit (blue dotted line) within the age range of the moving site, the model quickly diverges as the age deviates from the input data. Consequently, the model can be used to harmonized data within this age range, but should be used cautiously for data outside of it. The proposed hyperparameter tuning technique of Clinical ComBAT (Table 5) automatically increases the regularization parameters to limit deviation from the target model when no moving data is available. In panel B of FIG. 20, the technique is applied with τ₁=0.25, τ₂=4, yielding an optimal regularization parameter λ=1,278. In panel C of FIG. 20, with τ₁=1, τ₂=1, the technique yields an optimal regularization parameter λ=1e10. This effectively fixes the shape of the moving site curve to the target site curve, estimating only the intercept. Overall, data models estimated in panels B and C in FIG. 12 are suitable for the harmonization of moving site subjects of all ages.

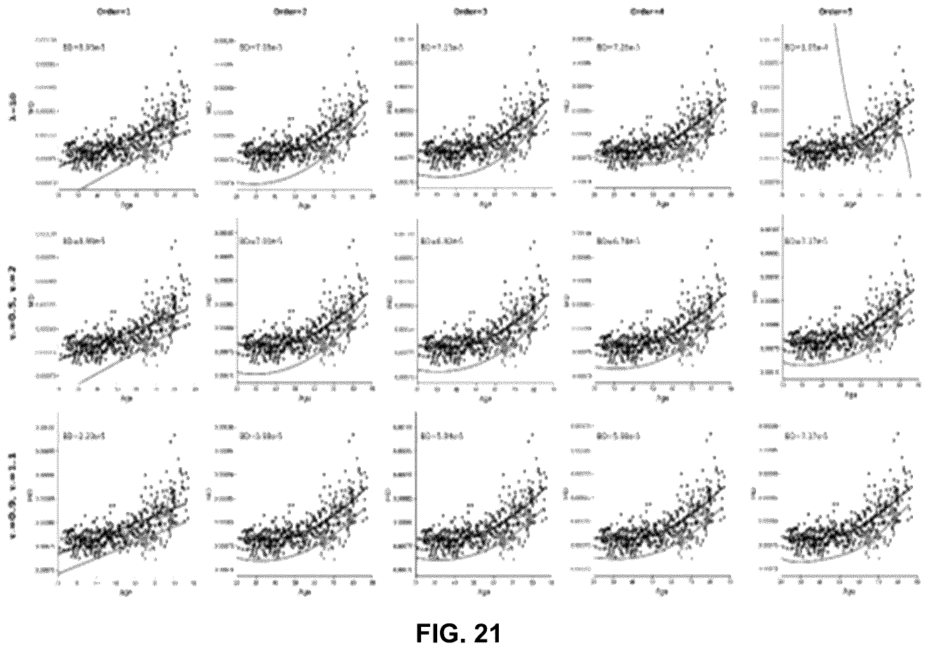

FIG. 21 presents a drawing of an example of a fit of mean diffusivity of the arcuate fasciculus in the left hemisphere using a Clinical ComBAT technique in accordance with an embodiment of the present disclosure. Notably, FIG. 21 highlights the effects of using reference site a priori with and without automatic hyperparameter tuning for various λ values with a fix ν₀=5 with data model of polynomial order P equal to 1 to 5. Note that the first row shows the fitted model using a fixed λ=10. The second and third rows show the fitted model using automatic hyperparameter tuning (τ₁=0.5 and τ₂=2, and τ₁=0.9 and τ₂=1.1, respectively). As can be seen, the use of a polynomial degree of 2 and 3 with an auto-tuning with τ₁=0.5, τ₂=2 yields the best results. High polynomial degrees (P equal to 4 and P equal to 5) need auto-tuning to be usable outside the age range of the input data.

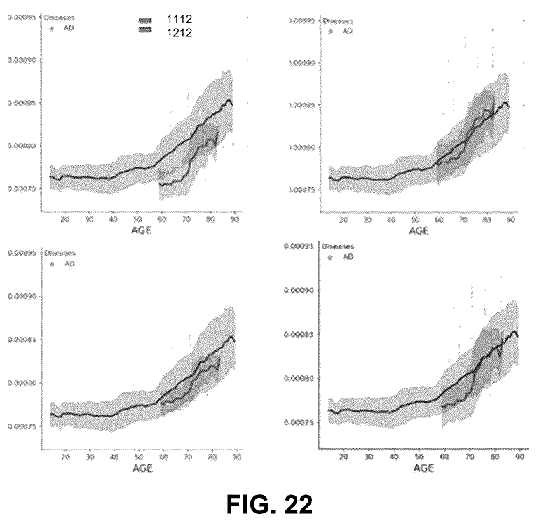

FIG. 22 presents a drawing of an example of outlier rejection in accordance with an embodiment of the present disclosure. Notably, FIG. 22 shows harmonization results obtained with the disclosed outlier rejection technique. On the top left, we see the target distribution in gray level containing only healthy controls and the unharmonized moving distribution in blue (from a site of the 1212 dataset) with Alzheimer's disease subjects as green dots. On the top right is the results of Clinical ComBAT when only the healthy controls of the moving site are considered. On the bottom left, is the harmonization of the moving site when both the healthy controls and the Alzheimer's disease subjects are considered. As a result, the healthy control blue distribution is poorly aligned with the target distribution and the Alzheimer's disease subjects falls inside the normative distribution. On the bottom right is the results of Clinical ComBAT harmonization when the outlier rejection technique is used to remove outliers (here Alzheimer's disease subjects). As can be seen, the result is close to that obtained with only healthy control with Alzheimer's disease subjects being above the normative distribution.

Table 8 summarizes the 11 features in Clinical ComBAT.

TABLE 8

| Feature | Clinical ComBAT |
|---|---|
| 1. | Pairwise harmonization between a moving site and a well populated target |
| | site |
| 2. | New data formation model, Eqn. 19 with one additive bias and one |
| | multiplicative bias |
| 3. | The model parameter vector \vec{β}ᵢᵥ is site specific |
| 4. | Use of a non-linear model in Eqns. 20 and 21 |
| 5. | $The variance of the target site {d}_{Tv}^{2} is computed with a maximum likelihood$ |
| | (Eqn. 24) |
| 6. | The model parameter vector of the moving site \vec{β}_{Mv} is computed with a |
| | maximum a posteriori formulation with the data of the moving site and a |
| | prior depending on the parameter vector of the target site \vec{β}_{Tv}. (Eqn. 28) |
| 7. | $The variance of the moving site {d}_{Mv}^{2} is computed with a maximum a$ |
| | posteriori formulation involving a Gamma prior distribution based at least |
| | $in part on the variance of the target site {d}_{Tv}^{2} ( Eqn . 32 )$ |
| 8. | Use of a quality control module that measures the goodness of fit between |
| | the target site and the harmonized moving site (Table 5) |
| 9. | Use of an outlier rejection module to remove outliers from the moving site |
| 10. | Some hyperparameters (like \vec{λ} and v₀) are automatically estimated |
| | following an auto-tuning technique (Table 6) |
| 11. | When the amount of data in the moving site is too low or that the data is |
| | poorly distributed across its co-variables, one can use Community- |
| | ComBAT to compensate for it (Table 7) |

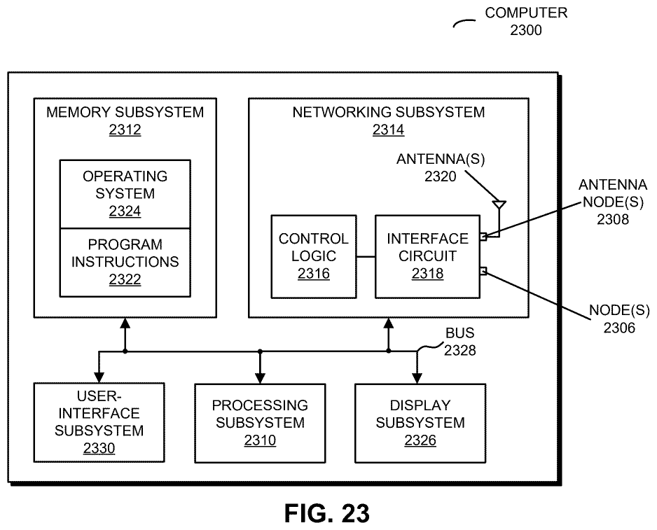

We now describe embodiments of a computer, which may perform at least some of the operations in the Clinical ComBAT techniques. FIG. 23 presents a block diagram illustrating an example of a computer 2300, e.g., in a computer system (such as computer system 200 in FIG. 2), in accordance with some embodiments. For example, computer 2300 may include: one of computers 210. This computer may include processing subsystem 2310, memory subsystem 2312, and networking subsystem 2314. Processing subsystem 2310 includes one or more devices configured to perform computational operations. For example, processing subsystem 2310 can include one or more microprocessors, ASICs, microcontrollers, programmable-logic devices, GPUs and/or one or more DSPs. Note that a given component in processing subsystem 2310 are sometimes referred to as a ‘computation device’.

Memory subsystem 2312 includes one or more devices for storing data and/or instructions for processing subsystem 2310 and networking subsystem 2314. For example, memory subsystem 2312 can include dynamic random access memory (DRAM), static random access memory (SRAM), and/or other types of memory. In some embodiments, instructions for processing subsystem 2310 in memory subsystem 2312 include: program instructions or sets of instructions (such as program instructions 2322 or operating system 2324), which may be executed by processing subsystem 2310. Note that the one or more computer programs or program instructions may constitute a computer-program mechanism. Moreover, instructions in the various program instructions in memory subsystem 2312 may be implemented in: a high-level procedural language, an object-oriented programming language, and/or in an assembly or machine language. Furthermore, the programming language may be compiled or interpreted, e.g., configurable or configured (which may be used interchangeably in this discussion), to be executed by processing subsystem 2310.

In addition, memory subsystem 2312 can include mechanisms for controlling access to the memory. In some embodiments, memory subsystem 2312 includes a memory hierarchy that comprises one or more caches coupled to a memory in computer 2300. In some of these embodiments, one or more of the caches is located in processing subsystem 2310.

In some embodiments, memory subsystem 2312 is coupled to one or more high-capacity mass-storage devices (not shown). For example, memory subsystem 2312 can be coupled to a magnetic or optical drive, a solid-state drive, or another type of mass-storage device. In these embodiments, memory subsystem 2312 can be used by computer 2300 as fast-access storage for often-used data, while the mass-storage device is used to store less frequently used data.

Networking subsystem 2314 includes one or more devices configured to couple to and communicate on a wired and/or wireless network (i.e., to perform network operations), including: control logic 2316, an interface circuit 2318 and one or more antennas 2320 (or antenna elements). (While FIG. 23 includes one or more antennas 2320, in some embodiments computer 2300 includes one or more nodes, such as antenna nodes 2308, e.g., a metal pad or a connector, which can be coupled to the one or more antennas 2320, or nodes 2306, which can be coupled to a wired or optical connection or link. Thus, computer 2300 may or may not include the one or more antennas 2320. Note that the one or more nodes 2306 and/or antenna nodes 2308 may constitute input(s) to and/or output(s) from computer 2300.) For example, networking subsystem 2314 can include a Bluetooth™ networking system, a cellular networking system (e.g., a 3G/4G/5G network such as UMTS, LTE, etc.), a universal serial bus (USB) networking system, a networking system based on the standards described in IEEE 802.11 (e.g., a Wi-Fi® networking system), an Ethernet networking system, and/or another networking system.

Networking subsystem 2314 includes processors, controllers, radios/antennas, sockets/plugs, and/or other devices used for coupling to, communicating on, and handling data and events for each supported networking system. Note that mechanisms used for coupling to, communicating on, and handling data and events on the network for each network system are sometimes collectively referred to as a ‘network interface’ for the network system. Moreover, in some embodiments a ‘network’ or a ‘connection’ between the electronic devices does not yet exist. Therefore, computer 2300 may use the mechanisms in networking subsystem 2314 for performing simple wireless communication between electronic devices, e.g., transmitting advertising or beacon frames and/or scanning for advertising frames transmitted by other electronic devices.

Within computer 2300, processing subsystem 2310, memory subsystem 2312, and networking subsystem 2314 are coupled together using bus 2328. Bus 2328 may include an electrical, optical, and/or electro-optical connection that the subsystems can use to communicate commands and data among one another. Although only one bus 2328 is shown for clarity, different embodiments can include a different number or configuration of electrical, optical, and/or electro-optical connections among the subsystems.

In some embodiments, computer 2300 includes a display subsystem 2326 for displaying information on a display, which may include a display driver and the display, such as a liquid-crystal display, a multi-touch touchscreen, etc. Moreover, computer 2300 may include a user-interface subsystem 2330, such as: a mouse, a keyboard, a trackpad, a stylus, a voice-recognition interface, and/or another human-machine interface.

Computer 2300 can be (or can be included in) any electronic device with at least one network interface. For example, computer 2300 can be (or can be included in): a desktop computer, a laptop computer, a subnotebook/netbook, a server, a supercomputer, a tablet computer, a smartphone, a cellular telephone, a consumer-electronic device, a portable computing device, communication equipment, and/or another electronic device.

Although specific components are used to describe computer 2300, in alternative embodiments, different components and/or subsystems may be present in computer 2300. For example, computer 2300 may include one or more additional processing subsystems, memory subsystems, networking subsystems, and/or display subsystems. Additionally, one or more of the subsystems may not be present in computer 2300. Moreover, in some embodiments, computer 2300 may include one or more additional subsystems that are not shown in FIG. 23. Also, although separate subsystems are shown in FIG. 23, in some embodiments some or all of a given subsystem or component can be integrated into one or more of the other subsystems or component(s) in computer 2300. For example, in some embodiments program instructions 2322 are included in operating system 2324 and/or control logic 2316 is included in interface circuit 2318.

Moreover, the circuits and components in computer 2300 may be implemented using any combination of analog and/or digital circuitry, including: bipolar, PMOS and/or NMOS gates or transistors. Furthermore, signals in these embodiments may include digital signals that have approximately discrete values and/or analog signals that have continuous values. Additionally, components and circuits may be single-ended or differential, and power supplies may be unipolar or bipolar.

An integrated circuit may implement some or all of the functionality of networking subsystem 2314 and/or computer 2300. The integrated circuit may include hardware and/or software mechanisms that are used for transmitting signals from computer 2300 and receiving signals at computer 2300 from other electronic devices. Aside from the mechanisms herein described, radios are generally known in the art and hence are not described in detail. In general, networking subsystem 2314 and/or the integrated circuit may include one or more radios.

In some embodiments, an output of a process for designing the integrated circuit, or a portion of the integrated circuit, which includes one or more of the circuits described herein may be a computer-readable medium such as, for example, a magnetic tape or an optical or magnetic disk or solid state disk. The computer-readable medium may be encoded with data structures or other information describing circuitry that may be physically instantiated as the integrated circuit or the portion of the integrated circuit. Although various formats may be used for such encoding, these data structures are commonly written in: Caltech Intermediate Format (CIF), Calma GDS II Stream Format (GDSII), Electronic Design Interchange Format (EDIF), OpenAccess (OA), or Open Artwork System Interchange Standard (OASIS). Those of skill in the art of integrated circuit design can develop such data structures from schematics of the type detailed above and the corresponding descriptions and encode the data structures on the computer-readable medium. Those of skill in the art of integrated circuit fabrication can use such encoded data to fabricate integrated circuits that include one or more of the circuits described herein.

While some of the operations in the preceding embodiments were implemented in hardware or software, in general the operations in the preceding embodiments can be implemented in a wide variety of configurations and architectures. Therefore, some or all of the operations in the preceding embodiments may be performed in hardware, in software or both. For example, at least some of the operations in the Clinical ComBAT techniques may be implemented using program instructions 2322, operating system 2324 (such as a driver for interface circuit 2318) or in firmware in interface circuit 2318. Thus, the Clinical ComBAT techniques may be implemented at runtime of program instructions 2322. Alternatively or additionally, at least some of the operations in the Clinical ComBAT techniques may be implemented in a physical layer, such as hardware in interface circuit 2318.

In the preceding description, we refer to ‘some embodiments’. Note that ‘some embodiments’ describes a subset of all of the possible embodiments, but does not always specify the same subset of embodiments. Moreover, note that the numerical values provided are intended as illustrations of the Clinical ComBAT techniques. In other embodiments, the numerical values can be modified or changed.

The foregoing description is intended to enable any person skilled in the art to make and use the disclosure, and is provided in the context of a particular application and its requirements. Moreover, the foregoing descriptions of embodiments of the present disclosure have been presented for purposes of illustration and description only. They are not intended to be exhaustive or to limit the present disclosure to the forms disclosed. Accordingly, many modifications and variations will be apparent to practitioners skilled in the art, and the general principles defined herein may be applied to other embodiments and applications without departing from the spirit and scope of the present disclosure. Additionally, the discussion of the preceding embodiments is not intended to limit the present disclosure. Thus, the present disclosure is not intended to be limited to the embodiments shown, but is to be accorded the widest scope consistent with the principles and features disclosed herein.

What is claimed is:

1. A computer system, comprising:
a computation device;
memory configured to store program instructions, wherein, when executed by the computation device, the program instructions cause the computer system to perform one or more operations comprising:
receiving information specifying magnetic-resonance diffusion measurements, wherein the magnetic-resonance diffusion measurements are associated with at least an individual and a first site;
performing pairwise harmonizing of the magnetic-resonance diffusion measurements based at least in part on target magnetic-resonance diffusion measurements, wherein the target magnetic-resonance diffusion measurements are associated with a population and one or more target sites, and
wherein the magnetic-resonance diffusion measurements and the target magnetic-resonance diffusion measurements are acquired using different magnetic-resonance scanners; and
performing quality control on the pairwise harmonized magnetic-resonance diffusion measurements, wherein the quality control comprises comparing the pairwise harmonized magnetic-resonance diffusion measurements associated with the first site and the one or more target sites, wherein the target magnetic-resonance diffusion measurements from the population are used as a prior for computing of parameters for a moving population associated with the first site.

2. The computer system of claim 1, wherein the magnetic-resonance diffusion measurements comprises measurements associated with the individual and a second individual.

3. The computer system of claim 1, wherein the magnetic-resonance diffusion measurements are associated with a different number of individuals than the target magnetic-resonance diffusion measurements.

4. The computer system of claim 1, wherein the quality control comprises detecting a harmonization error in the pairwise harmonized magnetic-resonance diffusion measurements based at least in part on the pairwise harmonized magnetic-resonance diffusion measurements and a quality-control metric; and
wherein the detecting comprises segmenting the pairwise harmonized magnetic-resonance diffusion measurements into a first type of magnetic-resonance diffusion result or a second type of magnetic-resonance diffusion result.

5. The computer system of claim 1, wherein the performing of the pairwise harmonization comprises eliminating one or more outliers in the magnetic-resonance diffusion measurements based at least in part on the pairwise harmonized magnetic-resonance diffusion measurements.

6. The computer system of claim 1, wherein the magnetic-resonance diffusion measurements comprise tractography results.

7. The computer system of claim 1, wherein the magnetic-resonance diffusion measurements comprise: fractional anisotropy, apparent fiber density, an amount of free water, a demyelination metric, one or more brain-region shape metric, a scalar map, or a scalar value.

8. The computer system of claim 1, wherein the pairwise harmonizing reduces or eliminates additive and multiplicative bias in the magnetic-resonance diffusion measurements.

9. The computer system of claim 1, wherein, after performing the pairwise harmonization, the operations comprise pooling the magnetic-resonance diffusion measurements and the target magnetic-resonance diffusion measurements.

10. The computer system of claim 1, wherein, after performing the pairwise harmonization, the operations comprise generating a normative reference based at least in part on the magnetic-resonance diffusion measurements and the target magnetic-resonance diffusion measurements.

11. The computer system of claim 1, wherein the magnetic-resonance diffusion measurements are associated with clinical operation of a magnetic-resonance scanner.

12. The computer system of claim 1, wherein the pairwise harmonization facilitates personalized medicine for at least the individual.

13. The computer system of claim 1, wherein the performing of the pairwise harmonization comprises:
fitting the magnetic-resonance diffusion measurements using a data model;
removing covariates and an intercept from the magnetic-resonance diffusion measurements using the data model;
after removing the covariates and the intercept, computing variance in the magnetic-resonance diffusion measurements;
after computing the variance, correcting the magnetic-resonance diffusion measurements for multiplicative bias; and
adding second covariates and a second intercept associated with the target magnetic-resonance diffusion measurements to the magnetic-resonance diffusion measurements.

14. The computer system of claim 13, wherein the data model comprises a nonlinear model.

15. The computer system of claim 13, wherein the variance is computed using maximum likelihood.

16. The computer system of claim 13, wherein the performing of the pairwise harmonization comprises hyper-parameter autotuning with the target magnetic-resonance diffusion measurements to prevent overfitting to the data model.

17. The computer system of claim 1, wherein the performing of the pairwise harmonization comprises supplementing the magnetic-resonance diffusion measurements with additional magnetic-resonance diffusion measurements from a different magnetic-resonance scanner when a number of individuals associated with the magnetic-resonance diffusion measurements is less than a predefined value; and
wherein the magnetic-resonance diffusion measurements and the additional magnetic-resonance diffusion measurements have characteristics within a predefined value of each other.

18. A non-transitory computer-readable storage medium for use in conjunction with a computer system, the computer-readable storage medium configured to store program instructions that, when executed by the computer system, causes the computer system to perform one or more operations comprising:
receiving information specifying magnetic-resonance diffusion measurements, wherein the magnetic-resonance diffusion measurements are associated with at least an individual and a first site;
performing pairwise harmonizing of the magnetic-resonance diffusion measurements based at least in part on target magnetic-resonance diffusion measurements, wherein the target magnetic-resonance diffusion measurements are associated with a population and one or more target sites, and
wherein the magnetic-resonance diffusion measurements and the target magnetic-resonance diffusion measurements are acquired using different magnetic-resonance scanners; and
performing quality control on the pairwise harmonized magnetic-resonance diffusion measurements, wherein the quality control comprises comparing the pairwise harmonized magnetic-resonance diffusion measurements associated with the first site and the one or more target sites, wherein the target magnetic-resonance diffusion measurements from the population are used as a prior for computing of parameters for a moving population associated with the first site.

19. A method for performing pairwise harmonization, comprising:
by a computer system:
receiving information specifying magnetic-resonance diffusion measurements, wherein the magnetic-resonance diffusion measurements are associated with at least an individual and a first site;
performing the pairwise harmonizing of the magnetic-resonance diffusion measurements based at least in part on target magnetic-resonance diffusion measurements, wherein the target magnetic-resonance diffusion measurements are associated with a population and one or more target sites, and
wherein the magnetic-resonance diffusion measurements and the target magnetic-resonance diffusion measurements are acquired using different magnetic-resonance scanners; and
performing quality control on the pairwise harmonized magnetic-resonance diffusion measurements, wherein the quality control comprises comparing the pairwise harmonized magnetic-resonance diffusion measurements associated with the first site and the one or more target sites, wherein the target magnetic-resonance diffusion measurements from the population are used as a prior for computing of parameters for a moving population associated with the first site.

20. The method of claim 19, wherein the method comprises detecting a harmonization error in the magnetic-resonance diffusion measurements based at least in part on the pairwise harmonizing and a quality-control metric; and
wherein the detecting comprises segmenting the magnetic-resonance diffusion measurements into a first type of magnetic-resonance diffusion result or a second type of magnetic-resonance diffusion result.
